(19) United States
(12) Patent Application Publication
(10) Pub. No.: US 2015/0045353 A1
(43) Pub. Date: Feb. 12, 2015

(54) FORMULATIONS CONTAINING GAMMA SECRETASE MODULATORS, METHODS FOR PREPARATION AND DELIVERY THEREOF

(71) Applicant: Neurogenetic Pharmaceuticals, Inc., Del Mar, CA (US)

(72) Inventors: William T. COMER, Del Mar, CA (US); Maria Z. KOUNNAS, Del Mar, CA (US); Bryan M. KNOX, San Diego, CA (US); Nathan S. BARKSDALE, San Diego, CA (US)

(73) Assignee: Neurogenetic Pharmaceuticals, Inc., Del Mar, CA (US)

(21) Appl. No.: 13/963,970

(22) Filed: Aug. 9, 2013

(51) Int. Cl. A61K 9/16; A61K 31/427; A61K 31/506; A61K 31/541; A61K 31/4545; A61K 31/4439; A61K 31/5377

(52) U.S. Cl. CPC A61K 9/16; A61K 31/4439; A61K 31/427; A61K 31/5377; A61K 31/541; A61K 31/4545; A61K 31/506; A61K 9/1694; A61K 9/1652
USPC 514/227.8; 546/270.4; 548/198; 544/124; 544/60; 546/194; 544/331; 514/341; 514/370; 514/235.8; 514/318; 514/275; 548/193

(57) ABSTRACT

In accordance with the present invention, there are provided formulations of gamma secretase modulators (GSMs) which are suitable for oral delivery and have improved transport properties relative to prior art formulations thereof. Also provided are methods for the preparation of such improved formulations and uses thereof for the delivery of GSMs to subjects in need thereof.

### FIELD OF THE INVENTION

The invention relates to formulations containing gamma secretase modulators, methods for the preparation of such formulations, and the use thereof for the delivery of gamma secretase modulators to a subject in need thereof. Invention formulations are useful for the treatment of a variety of neurological disorders.

### BACKGROUND OF THE INVENTION

The information provided herein and references cited are provided solely to assist the understanding of the reader, and does not constitute an admission that any of the references or information is prior art to the present invention.

Neurodegenerative diseases are disorders characterized by destruction or deterioration of selective neuronal populations. Exemplary neurodegenerative diseases include Alzheimer's disease (AD), Parkinsonian syndromes such as (PD), Huntington's disease (HD), Prion diseases, cerebral amyloid angiopathy (CAA), and mild cognitive impairment (MCI). Neurodegenerative disease is associated with progressive nervous system dysfunction, and often leads to atrophy of affected central or peripheral nervous system structures.

Alzheimer's disease (AD) is a progressive neurodegenerative disorder that is the predominant cause of dementia in people over 65 years of age. Clinical symptoms of the disease begin with subtle short-term memory problems. As the disease progresses, difficulty with memory, language and orientation worsen to the point of interfering with the ability of the person to function independently. Other symptoms, which are variable, include myoclonus and seizures. Duration of AD from the first symptoms of memory loss until death is 10 years on average.

AD is characterized by massive neuronal cell loss in certain brain areas, and by the deposition of proteinaceous material in the brains of AD patients. These deposits contain neurofibrillary tangles and β-amyloid plaques. The major protein component of the β-amyloid plaque is Aβ.

Increased accumulation of Aβ has been postulated to significantly contribute to the pathogenesis of AD, and is also associated with various other amyloidoses and neurological disorders, such as Parkinson's disease, Down syndrome, diffuse Lewy body disease, progressive supranuclear palsy, and Hereditary Cerebral Hemorrhage with Amyloidosis-Dutch Type (HCHWA-D), cerebral amyloid angiopathy (CAA), and mild cognitive impairment (MCI). Support for a role for Aβ in AD can be found in Down patients who develop AD-like symptoms and pathology after age 40. Such patients exhibit AD-like amyloid plaques prior to the onset of other AD symptoms, suggesting that increased amyloid accumulation is an initial pathogenic event. Additional evidence implicating accumulation of Aβ peptides in AD comes from the identification of various mutations that result in increased formation of Aβ by cells that account for certain types of inherited AD (familial AD, or FAD). FAD individuals comprise 10% of all AD cases and generally exhibit symptoms of the disease much earlier than sporadic AD patients.

Aβ peptides are derived from processing of an amyloid precursor protein (APP). mRNA generated from the APP gene on chromosome 21 undergoes alternative splicing to yield about 10 possible isoforms, three of which (APP695, 751, and 770 amino acid isoforms) predominate in the brain. APP695 is the shortest of the three isoforms and is produced mainly in neurons. APP751, which contains a Kunitz-protease inhibitor (KPI) domain, and APP770, which contains both the KPI domain and an MRC-OX2 antigen domain, are found mostly in non-neuronal glial cells.

The major APP isoforms are single-transmembrane proteins, composed of an extracellular amino-terminal domain (approximately 590-680 amino acids) and a cytoplasmic tail containing intracellular trafficking signals (approximately 55 amino acids). Within APP, the Aβ peptide sequence is located partially on the extracellular side of the membrane and extends partially into the transmembrane region. APP isoforms 695, 751, and 770 share the same Aβ, transmembrane, and intracellular domains.

APP is trafficked through the constitutive secretory pathway, where it undergoes post-translational processing, including cleavage via two pathways: an amyloidogenic pathway and a non-amyloidogenic pathway. In the non-amyloidogenic pathway, APP is cleaved by α-secretase within the Aβ domain, releasing a large soluble N-terminal fragment (sAPPα) for secretion and a non-amyloidogenic C-terminal fragment (C83). Because cleavage occurs within the Aβ domain, α-secretase cleavage in the non-amyloidogenic pathway precludes Aβ formation. The C-terminal fragment of APP generated by α-secretase cleavage (C83) is subsequently cleaved by γ-secretase within the predicted transmembrane domain to generate a non-amyloidogenic peptide fragment termed p3 (22-24 residues).

In the amyloidogenic pathway, APP is cleaved by β-secretase (BACE1 or BACE2 enzymes) at the beginning of the Aβ domain that defines the amino terminus of the Aβ peptide. Cleavage by BACE1 or BACE2 generates a shorter soluble N-terminus, sAPPβ, as well as an amyloidogenic C-terminal fragment (C99). Alternatively, BACE1 can also cleave APP 10 amino acids after the beginning of the Aβ domain (between amino acid 10 and 11) to generate a longer N-terminal soluble fragment and a shorter C-terminal fragment (C89). Additional cleavage of either C89 or C99 by γ-secretase, a presenilin-dependent enzyme, produces Aβ peptides of various lengths.

The predominant forms of Aβ found in plaques from AD brains are the Aβ42 and Aβ40 species. Aβ42 is the species initially deposited in brain plaques, and is highly prone to aggregation in vitro. Therefore, the Aβ42 species of amyloid peptide, in particular, may be a viable target in the development of therapeutics for the treatment of disease or disorders characterized by Aβ accumulation.

Currently, there is no cure or effective treatment for AD, and the few approved drugs, including Aricept, Exelon, Cognex and Reminyl, are palliative at best. Based on the correlation between Aβ accumulation, neuronal loss and AD, modulating Aβ levels, such as reducing levels of pathogenic Aβ species, represents a viable way to decrease plaque formation and minimize neuronal cell death. Thus, there exists a medical need for compounds that modulate levels of Aβ, and effective methods for the delivery thereof, especially in view of the challenges in delivery of pharmacologically active compounds across the blood-brain barrier. Indeed, such compounds, and formulations containing same, would be useful for the treatment of neurodegenerative disorders, such as AD.

### SUMMARY OF THE INVENTION

In accordance with the present invention, there are provided formulations of gamma secretase modulators (GSMs) which are suitable for oral delivery and have improved transport properties relative to prior art formulations thereof. Also provided are methods for the preparation of such improved formulations and uses thereof for the delivery of GSMs to subjects in need thereof.

### BRIEF DESCRIPTION OF THE FIGURES

FIG. 1 shows the dissolution profile for each formulation referred to in Example 2.

FIG. 2 shows the dissolution profile for each formulation referred to in Example 4.

FIG. 3 shows the dissolution profile for several formulations as a function of gelucire concentration (see Example 6).

FIG. 4 shows the dissolution results for the formulations described in Example 10.

### DETAILED DESCRIPTION OF THE INVENTION

In accordance with the present invention, there are provided formulations suitable for delivery of a gamma secretase modulator (GSM) to a subject in need thereof, said formulation comprising one or more of (a), (b), (c) and/or (d) as follows:
(a) a finely divided form of said GSM; and/or
(b) a substantially amorphous form of said GSM; and/or
(c) one or more GSM(s) in the further presence of one or more excipients therefor; and/or
(d) any of (a), (b) or (c) above, in combination with food; administration of such dosage forms in combination with food further enhances the absorption and bioavailability of said GSM.

Invention formulations optionally further comprise one or more excipients as are well known to those skilled in the formulation art. Therefore, in accordance with an aspect of the present invention, any excipient or combination of excipients known in the pharmaceutical art may be used. Examples may include flow aids, stabilizers, anti-oxidants, surface active agents, binders, dispersing agents, a disintegrant (e.g., croscarmellose sodium, sodium starch glycolate, cross-linked povidone, and the like), flavorings, taste masking agents, coatings, release control agents, water, and/or other excipients typically employed for formulation of oral dosage forms. In some embodiments, the excipient may comprise one or more materials selected from the group consisting of microcrystalline cellulose, dicalcium phosphate, lactose, pre-gelatinized starch, carnauba wax, candelilla wax, silica, and magnesium stearate.

In some embodiments, compositions will comprise pharmaceutically acceptable carriers or excipients, such as fillers, binders, disintegrants, glidants, lubricants, complexing agents, solubilizers, surfactants, and the like, which may be chosen to facilitate administration of the compound by a particular route. Examples of carriers include calcium carbonate, calcium phosphate, various sugars such as lactose, glucose, or sucrose, types of starch, cellulose derivatives, gelatin, lipids, liposomes, nanoparticles, and the like. Carriers also include physiologically compatible liquids as solvents or for suspensions, including, for example, sterile solutions of water for injection (WFI), saline solution, dextrose solution, Hank's solution, Ringer's solution, vegetable oils, mineral oils, animal oils, polyethylene glycols, liquid paraffin, and the like. Excipients may also include, for example, colloidal silicon dioxide, silica gel, talc, magnesium silicate, calcium silicate, sodium aluminosilicate, magnesium trisilicate, powdered cellulose, macrocrystalline cellulose, carboxymethyl cellulose, cross-linked sodium carboxymethylcellulose, sodium benzoate, calcium carbonate, magnesium carbonate, stearic acid, aluminum stearate, calcium stearate, magnesium stearate, zinc stearate, sodium stearyl fumarate, syloid, stearowet C, magnesium oxide, starch, sodium starch glycolate, glyceryl monostearate, glyceryl dibehenate, glyceryl palmitostearate, hydrogenated vegetable oil, hydrogenated cotton seed oil, castor seed oil mineral oil, polyethylene glycol (e.g. PEG 4000-8000), polyoxyethylene glycol, poloxamers, povidone, crospovidone, croscarmellose sodium, alginic acid, casein, methacrylic acid divinylbenzene copolymer, sodium docusate, cyclodextrins (e.g. 2-hydroxypropyl-.delta.-cyclodextrin), polysorbates (e.g. polysorbate 80), cetrimide, TPGS (d-alpha-tocopheryl polyethylene glycol 1000 succinate), magnesium lauryl sulfate, sodium lauryl sulfate, polyethylene glycol ethers, di-fatty acid ester of polyethylene glycols, or a polyoxyalkylene sorbitan fatty acid ester (e.g., polyoxyethylene sorbitan ester Tween®), polyoxyethylene sorbitan fatty acid esters, sorbitan fatty acid ester, e.g. a sorbitan fatty acid ester from a fatty acid such as oleic, stearic or palmitic acid, mannitol, xylitol, sorbitol, maltose, lactose, lactose monohydrate or lactose spray dried, sucrose, fructose, calcium phosphate, dibasic calcium phosphate, tribasic calcium phosphate, calcium sulfate, dextrates, dextran, dextrin, dextrose, cellulose acetate, maltodextrin, simethicone, polydextrosem, chitosan, gelatin, HPMC (hydroxypropyl methyl celluloses), HPC (hydroxypropyl cellulose), hydroxyethyl cellulose, hypromellose, and the like.

Gamma secretase modulators (GSMs) contemplated for use herein include compounds of Formula (I) having the structure:

(A)-L_{A}-(B)-L_{B}-(C)-L_{C}-(D) (I)
as well as analogs, homologs, prodrugs, derivatives, and pharmaceutically acceptable salts thereof,
   wherein:
A is an optionally substituted 1,3-imidazole or an optionally substituted 1,2,3-triazole having the structure:

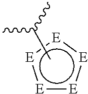

wherein E at positions 1 and 3 are N, E at position 2 is N or CH, E at position 4 is CR¹, and E at position 5 is CH;
each R¹ is hydrogen, halogen, substituted or unsubstituted alkyl, substituted or unsubstituted alkenyl, substituted or unsubstituted alkynyl, substituted or unsubstituted alkoxy, substituted or unsubstituted alkylamido, substituted or unsubstituted alkylamino, substituted or unsubstituted amino, substituted or unsubstituted cycloalkyl, or substituted or unsubstituted aryl;
B is an optionally substituted phenyl, an optionally substituted pyridyl or an optionally substituted pyrimidinyl having the structure:

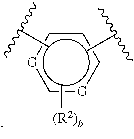

wherein each G is independently CR² or N;
each R² is independently selected from hydrogen, halogen, substituted or unsubstituted alkyl, substituted or unsubstituted alkenyl, substituted or unsubstituted alkynyl, substituted or unsubstituted alkoxy, substituted or unsubstituted alkylamido, substituted or unsubstituted alkylamino, or substituted or unsubstituted amino; and
b is 0-2;
C is an optionally substituted thiazole having the structure:

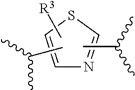

wherein R³ is hydrogen, halogen, substituted or unsubstituted alkyl, substituted or unsubstituted cycloalkyl, substituted or unsubstituted aryl, substituted or unsubstituted heteroaryl, or substituted or unsubstituted alkoxy;
D is an optionally substituted aryl having the structure:

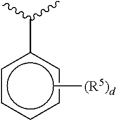

wherein each R⁵ is independently selected from hydrogen, halogen, substituted or unsubstituted alkyl, substituted or unsubstituted alkenyl, substituted or unsubstituted alkynyl, substituted or unsubstituted alkoxy, substituted or unsubstituted cycloalkyl, substituted or unsubstituted heterocycle, substituted or unsubstituted aryl, substituted or unsubstituted heteroaryl, or substituted or unsubstituted amino, or substituted or unsubstituted alkylamino; and
d is 0-5;
L_{A} is a covalent bond;
L_{B} is a covalent bond; and
L_{C} is —NR—.

As used herein, reference to a certain element such as hydrogen or H is meant to include all isotopes of that element. For instance, if a group is defined to include hydrogen or H, it also can include deuterium and/or tritium. In the structures provided herein, where a nitrogen atom appears to be divalent, it is assumed that the nitrogen atom is trivalent and the third substituent is hydrogen.

Compounds of the present invention may have asymmetric centers and may occur, except when specifically noted, as mixtures of stereoisomers or as individual diastereomers, or enantiomers, with all isomeric forms being included in the present invention. Compounds of the present invention embrace all conformational isomers. Compounds of the present invention may also exist in one or more tautomeric forms, including both single tautomers and mixtures of tautomers.

The phrase “hydrocarbyl” refers to any organic radical having a directly attachable carbon atom to any molecule presented herein. The phrase “substituted hydrocarbyl” refers to a hydrocarbyl group that is substituted according to the definition provided below. Hydrocarbyl groups include saturated and unsaturated hydrocarbons, straight and branched chain aliphatic hydrocarbons, cyclic hydrocarbons, and aromatic hydrocarbons.

The phrase “substituted” refers to an atom or group of atoms that has been replaced with another substituent. The phrase “substituted” includes any level of substitution, e.g. mono-, di-, tri-, tetra-, or penta-substitution, where such substitution is chemically permissible. Substitutions can occur at any chemically accessible position and on any atom, such as substitution(s) on carbons or any heteroatom. For example, substituted compounds are those where one or more bonds to a hydrogen or carbon atom(s) contained therein are replaced by a bond to non-hydrogen and/or non-carbon atom(s).

The phrase “alkyl” refers to hydrocarbyl chains comprising from 1 to 20 carbon atoms. The phrase “alkyl” includes straight chain alkyl groups, such as methyl, ethyl, propyl, butyl, pentyl, hexyl, heptyl, octyl, nonyl, decyl, undecyl, dodecyl, and the like. The phrase also includes branched chain isomers of straight chain alkyl groups, including but not limited to, the following which are provided by way of example: —CH(CH₃)₂, —CH(CH₃)(CH₂CH₃), —CH(CH₂CH₃)₂, —C(CH₃)₃, —C(CH₂CH₃)₃, —CH₂CH(CH₃)₂, —CH₂CH(CH₃)(CH₂CH₃), —CH₂CH(CH₂CH₃)₂, —CH₂C(CH₃)₃, —CH₂C(CH₂CH₃)₃, —CH(CH₃)CH(CH₃)(CH₂CH₃), —CH₂CH₂CH(CH₃)₂, —CH₂CH₂CH(CH₃)(CH₂CH₃), —CH₂CH₂CH(CH₂CH₃)₂, —CH₂CH₂C(CH₃)₃, —CH₂CH₂C(CH₂CH₃)₃, —CH(CH₃)CH₂CH(CH₃)₂, —CH(CH₃)CH(CH₃)CH(CH₃)₂, and —CH(CH₂CH₃)CH(CH₃)CH(CH₃)(CH₂CH₃). Thus, alkyl groups include primary alkyl groups, secondary alkyl groups, and tertiary alkyl groups. Preferred alkyl groups include alkyl groups having from 1 to 16 carbon atoms, or from 1 to 3 carbon atoms, such as methyl, ethyl, propyl, and —CH(CH₃)₂.

The phrase “substituted alkyl” refers to an alkyl group that is substituted according to the definition provided above. Examples of “substituted alkyl” groups include, but are not limited to, replacements of carbon or hydrogen atom(s) with a halogen atom(s), such as trifluoromethyl; an oxygen atom(s) in groups such as hydroxyl groups, alkoxy groups, aryloxy groups, and ester groups; a sulfur atom in groups such as thiol groups, alkyl and aryl sulfide groups, sulfone groups, sulfonyl groups, and sulfoxide groups; a nitrogen atom in groups such as amines, amides, alkylamines, dialkylamines, N-alkyloxides, imides, and enamines; a silicon atom in groups such as in trialkylsilyl groups, dialkylarylsilyl groups, alkyldiarylsilyl groups, and triarylsilyl groups; and other various heteroatoms. Additionally, substituted alkyl groups may be bonded to one or more carbon atom(s).

The phrase “alkenyl” refers to hydrocarbyl chains comprising from 2 to 20 carbon atoms and comprising at least one carbon-carbon double bond (—C═C—). The phrase “alkenyl” includes straight chain alkenyl groups, as well as branched chain isomers of straight chain alkenyl groups. Preferably, alkenyl groups comprise from 1 to 8 double bond(s). The phrase “substituted alkenyl” refers to an alkenyl group that is substituted according to the definition provided above.

The phrase “alkynyl” refers to hydrocarbyl chains comprising from 2 to 20 carbon atoms and comprising at least one carbon-carbon triple bond (—C═C—). The phrase “alkynyl” includes straight chain alkynyl groups, as well as branched chain isomers of straight chain alkynyl groups. Preferably, alkynyl groups comprise from 1 to 8 triple bond(s). The phrase “substituted alkynyl” refers to an alkynyl group that is substituted according to the definition provided above.

The phrase “cycloalkyl” refers to an alicyclic moiety having 3 to 20 carbon atoms and comprising any chemically permissible amount of saturated or unsaturated bonds. Preferably, cycloalkyl groups comprise from 4 to 7 carbons atoms. Cycloalkyl groups include, but are not limited to, cyclopropyl, cyclobutyl, cyclopentyl, cyclohexyl, cycloheptyl, cyclooctyl, and the like. The phrase “substituted cycloalkyl” refers to a cycloalkyl group that is substituted according to the definition provided above. Substituted cycloalkyl groups can have one or more atom substituted with straight or branched chain alkyl groups and can further comprise cycloalkyl groups that are substituted with other rings including fused rings. Examples of cycloalkyl groups that are substituted with fused rings include, but are not limited to, adamantyl, norbornyl, bicyclo[2.2.2]octyl, decalinyl, tetrahydronaphthyl, and indanyl, bornyl, camphenlyl, isocamphenyl, and carenyl groups. Representative substituted cycloalkyl groups may be mono-substituted or substituted more than once, such as, but not limited to, 2,2-, 2,3-, 2,4-, 2,5-, or 2,6-disubstituted cyclohexyl groups or mono-, di- or tri-substituted norbornyl or cycloheptyl groups, which may be substituted with, for example, alkyl, alkoxy, amino, thio, or halo groups.

The phrase “cycloalkylene” refers to divalent cycloalkyl groups comprising from 3 to 20 carbon atoms, and “substituted cycloalkylene” refers to cycloalkylene groups further bearing one or more substituents as set forth above.

The phrase “heterocyclyl”, “heterocyclic”, or “heterocycle” refers to nonaromatic cyclic hydrocarbyl compounds of which at least one ring member is a heteroatom. Heterocyclic groups include monocyclic, bicyclic, and polycyclic ring compounds containing from 3 to 20 ring members of which one or more is a heteroatom such as, but not limited to, N, O, and S. Heterocyclic groups include, any level of saturation. For instance, heterocyclic groups include unsaturated 3 to 8 membered rings containing 1 to 4 nitrogen atoms; saturated 3 to 8 membered rings containing 1 to 4 nitrogen atoms; condensed unsaturated heterocyclic groups containing 1 to 4 nitrogen atoms; unsaturated 3 to 8 membered rings containing 1 to 2 oxygen atoms and 1 to 3 nitrogen atoms; saturated 3 to 8 membered rings containing 1 to 2 oxygen atoms and 1 to 3 nitrogen atoms; unsaturated condensed heterocyclic groups containing 1 to 2 oxygen atoms and 1 to 3 nitrogen atoms; unsaturated 3 to 8 membered rings containing 1 to 3 sulfur atoms and 1 to 3 nitrogen atoms. Preferred heterocyclyl groups contain 5 or 6 ring members. Examples of heterocyclic groups include, but are not limited to, morpholine and piperazine. The phrase “substituted heterocyclyl” or “substituted heterocyclic” refers to a heterocyclyl group that is substituted according to the definition provided above.

The phrase “heterocyclene” or “heterocyclylene” refers to divalent heterocyclic (i.e., ring-containing) groups comprising from 3 to 20 carbon atoms and “substituted heterocycloalkylene” refers to heterocycloalkylene groups further bearing one or more substituents as set forth above.

The phrase “aryl” refers to single-ring aromatic radicals which may include from 5 to 20 carbon atoms. Aryl groups include, but are not limited to, phenyl, biphenyl, anthracenyl, and naphthenyl. The phrase “substituted aryl group” refers to an aryl group that is substituted according to the definition provided above. For example, substituted aryl groups may be bonded to one or more carbon atom(s), oxygen atom(s), nitrogen atom(s), and/or sulfur atom(s) and also includes aryl groups in which one or more aromatic carbons of the aryl group is bonded to a substituted and/or unsubstituted alkyl, alkenyl, or alkynyl group. This includes bonding arrangements in which two carbon atoms of an aryl group are bonded to two atoms of an alkyl, alkenyl, or alkynyl group to define a fused ring system (e.g. dihydronaphthyl or tetrahydronaphthyl). Thus, the phrase “substituted aryl” includes, but is not limited to tolyl, hydroxyphenyl, and the like.

The phrase “arylene” refers to divalent aryl groups comprising from 3 to 20 carbon atoms and “substituted arylene” refers to arylene groups further bearing one or more substituents as set forth above.

The phrase “heteroaryl” refers to a 3 to 20-membered aromatic ring consisting of carbon atoms and heteroatoms, such as N, S, and O or (ii) an 8- to 10-membered bicyclic or polycyclic ring system containing carbon atoms and heteroatoms, such as N, S, and O, wherein at least one of the rings in the bicyclic system is an aromatic ring. The heteroaryl ring may be attached at any heteroatom or carbon atom. Representative heteroaryl compounds include, for example, imidazolyl, pyridyl, pyrazinyl, pyrimidinyl, thiophenyl, thiazolyl, furanyl, pyridofuranyl, pyrimidofuranyl, pyridothienyl, pyridazothienyl, pyridooxazolyl, pyridazooxazolyl, pyrimidooxazolyl, pyridothiazolyl, pyridazothiazolyl, pyrrolyl, pyrrolinyl, imidazolyl, pyrazolyl, pyridyl, dihydropyridyl, pyrimidyl, pyrazinyl, pyridazinyl, triazolyl (e.g. 4H-1,2,4-triazolyl, 1H-1,2,3-triazolyl, and 2H-1,2,3-triazolyl), tetrazolyl, (e.g. 1H-tetrazolyl and 2H tetrazolyl), pyrrolidinyl, imidazolidinyl, piperidinyl, piperazinyl, indolyl, isoindolyl, indolinyl, indolizinyl, benzimidazolyl, quinolyl, isoquinolyl, indazolyl, benzotriazolyl, oxazolyl, isoxazolyl, oxadiazolyl (e.g. 1,2,4-oxadiazolyl, 1,3,4-oxadiazolyl, and 1,2,5-oxadiazolyl), benzoxazolyl, benzoxadiazolyl, benzoxazinyl (e.g. 2H-1,4-benzoxazinyl), thiazolyl, isothiazolyl, thiadiazolyl (e.g. 1,2,3-thiadiazolyl, 1,2,4-thiadiazolyl, 1,3,4-thiadiazolyl, and 1,2,5-thiadiazolyl). The phrase “substituted heteroaryl” refers to a heteroaryl group that is substituted according to the definition provided above.

The phrase “heteroarylene” refers to divalent aryl groups containing one or more heteroatoms (e.g., N, O, S or the like) as part of the aromatic ring, and typically having in the range of 3 up to 20 carbon atoms and “substituted heteroarylene” refers to heteroarylene groups further bearing one or more substituents as set forth above.

The phrase “alkoxy” refers to an oxygen-containing alkyl or cycloalkyl group, as defined above.

The phrase “alkylamido” refers to an alkyl group, as defined as above, which comprises —C(O)NR₂ wherein each R is independently hydrogen, alkyl, cycloalkyl, aryl, heteroaryl, or the like. Furthermore, alkylamido embraces embodiments wherein R, together with N, forms a cyclic structure.

The phrase “amino” refers to —NR₂ wherein each R is independently hydrogen, alkyl, cycloalkyl, aryl, heteroaryl, and the like. Furthermore, amino embraces embodiments wherein R, together with N, forms a cyclic structure.

The phrase “alkylamino” refers to an alkyl group, as defined as above, which comprises an amino group, as defined above.

The phrase “halogen” refers to F, Cl, Br, or I.

As used herein, “analogs” refers to variants of compounds described herein having a slightly altered chemical structure, e.g., as a result of the replacement of one ring structure with another, or by the introduction of a substituent at a previously unsubstituted site, or by the replacement of one substituent with another substituent.

As used herein, “homologs” refers to a compound which is part of a series of compounds differing from each other by a repeating unit, such as a methylene moiety (—CH₂—). A homolog is a special case of an analog.

As used herein, “prodrugs” refers to a compound that, upon in vivo administration, is metabolized by one or more steps or processes or otherwise converted to the biologically, pharmaceutically or therapeutically active form of the compound. Prodrugs can be prepared by modifying functional groups present in the compound in such a way that the modifications are cleaved, either in routine manipulation or in vivo, to a compound described herein. For example, prodrugs include compounds of the present invention wherein a hydroxy, amino, or sulfhydryl group is bonded to any group that, when administered to a mammalian subject, can be cleaved to form a free hydroxyl, free amino, or free sulfhydryl group, respectively. Representative prodrugs include, for example, esters, enol ethers, enol esters, acetates, formates, benzoate derivatives, and the like of alcohol and amine functional groups in the compounds of the present invention. By virtue of knowledge of pharmacodynamic processes and drug metabolism in vivo, those of skill in this art, once a pharmaceutically active compound is known, can design prodrugs of the compound (see, e.g., Nogrady (1985) Medicinal Chemistry A Biochemical Approach, Oxford University Press, New York, pages 388-392).

As used herein, “derivatives” refers to a compound that is derived from a similar compound by some chemical or physical process. Derivatives and chemically similar compounds within the scope of the instant disclosure may be prepared by routine modification of the procedures provided herein using the appropriate starting materials, the selection of which will be evident to those of skill in the art.

In certain embodiments of the present invention, ring A of compounds of Formula (I) has the structure:

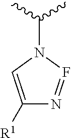

wherein F is CH or N. In certain embodiments, ring A of compounds of Formula (I) include compounds where R¹ is halogen or substituted or unsubstituted C₁-C₅ alkyl.

In certain embodiments of the present invention, ring B of compounds of Formula (I) has the structure:

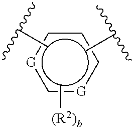

and b is 0-2. For instance, embodiments of B may be selected from

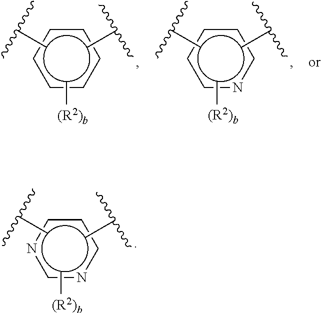

More preferable embodiments include compounds of Formula (I) wherein B is selected from

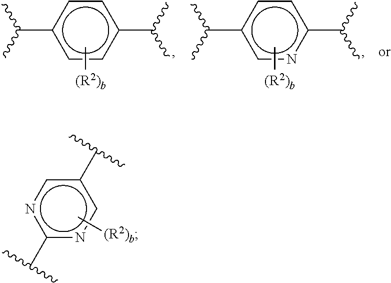

wherein each R² is independently selected from halogen or substituted or unsubstituted C₁-C₅ alkyl.

In certain embodiments of the present invention, ring C of compounds of Formula (I) have the structure

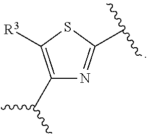

In certain embodiments of the present invention, ring D of compounds of Formula (I) has the structure

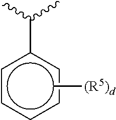

and d is 0-5, wherein each R⁵, when present, is independently selected from halogen, substituted or unsubstituted C₁-C₅ alkyl, substituted or unsubstituted C₁-C₅ alkoxy, substituted or unsubstituted five to six-membered heteroaryl, or N(R″)₂ wherein each R″ is independently a substituted or unsubstituted C₁-C₅ alkyl or C₃-C₁₀ cycloalkyl. In certain embodiments D is selected from

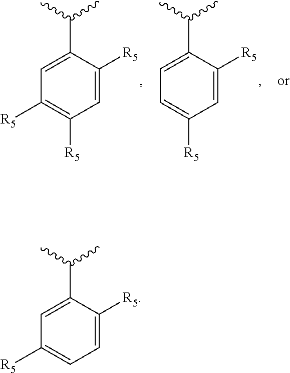

Exemplary embodiments contemplated herein include compounds having a structure corresponding to Formula (II):

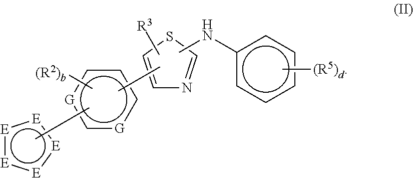

Further exemplary embodiments contemplated herein include compounds having a structure corresponding to Formula (III):

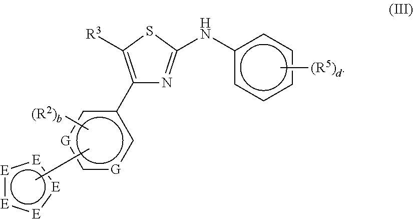

Additional exemplary embodiments contemplated herein include compounds having a structure corresponding to Formula (IV):

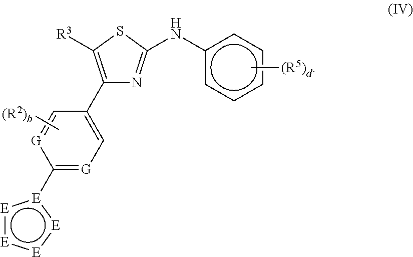

Still further exemplary embodiments contemplated herein include compounds having a structure corresponding to Formula (V):

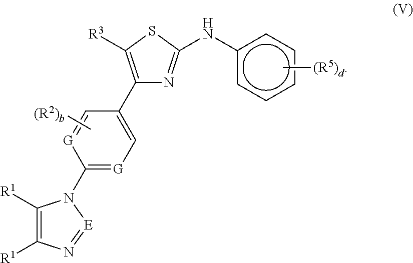

Exemplary compounds contemplated for use herein include:

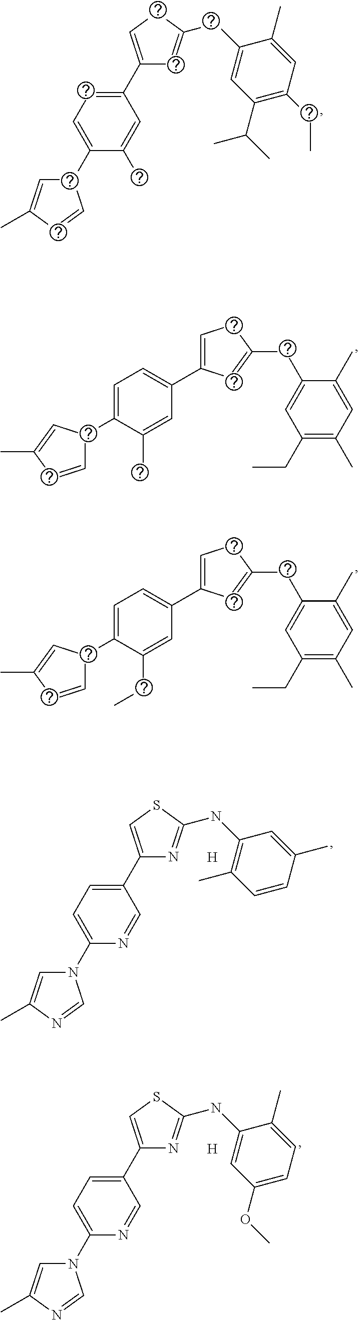

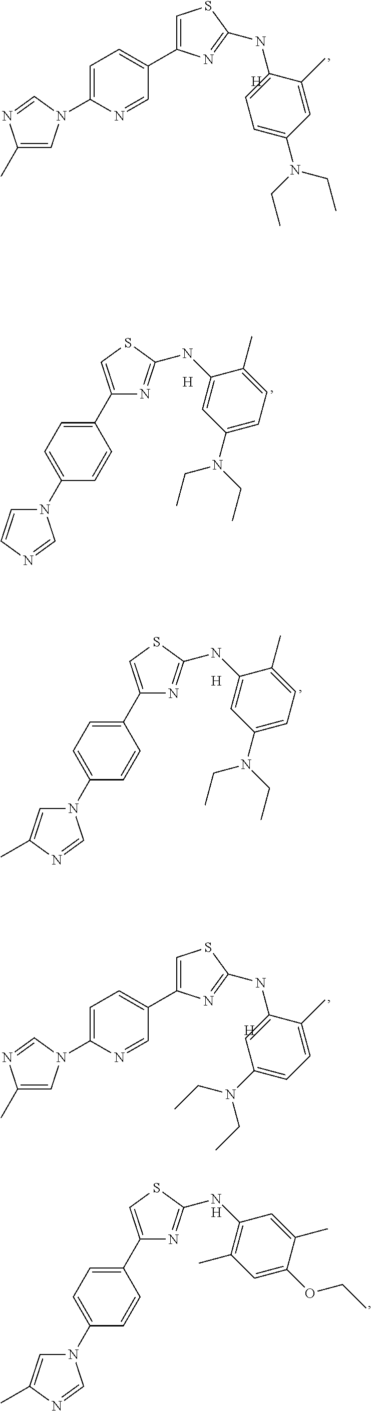

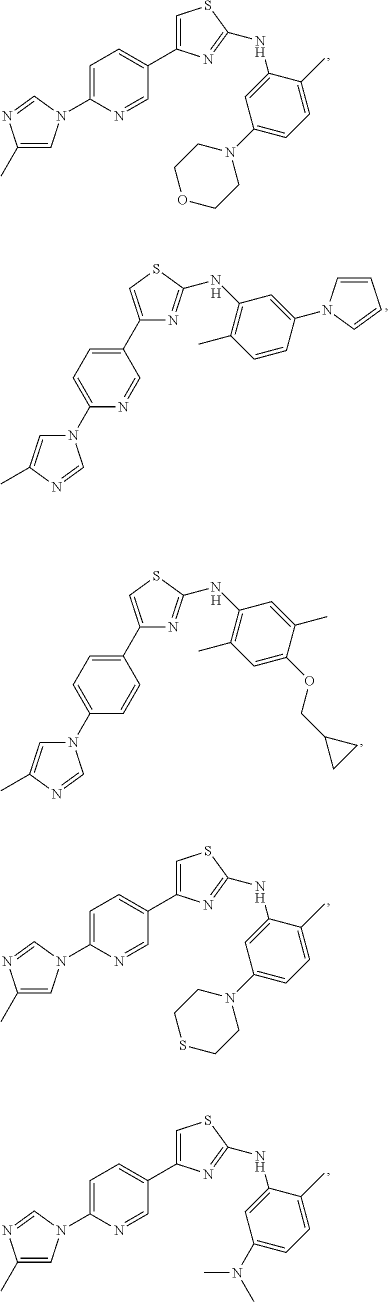

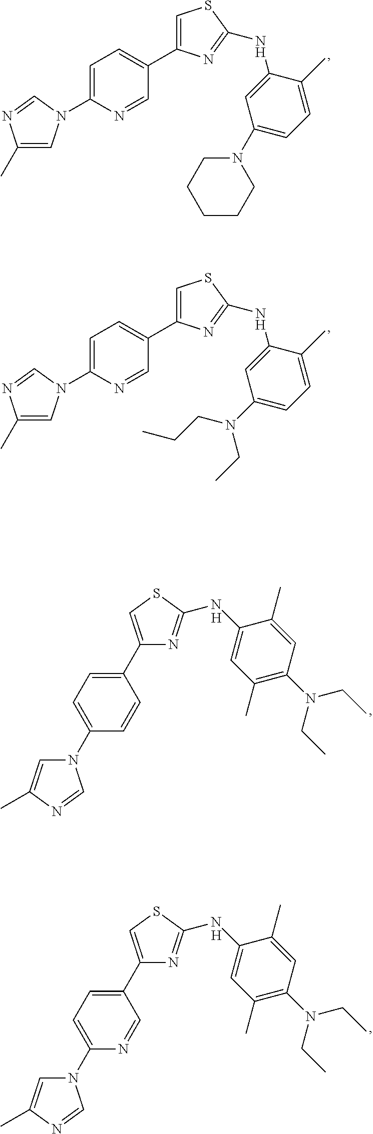

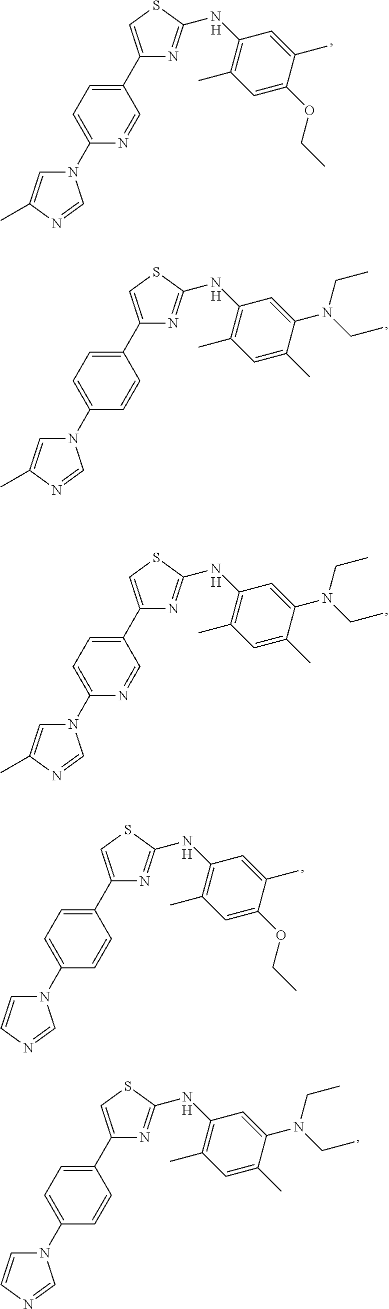

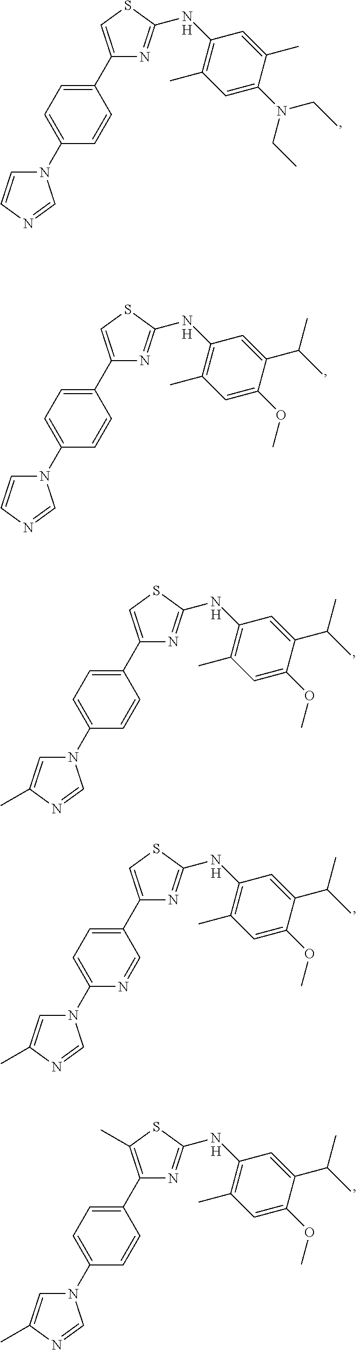

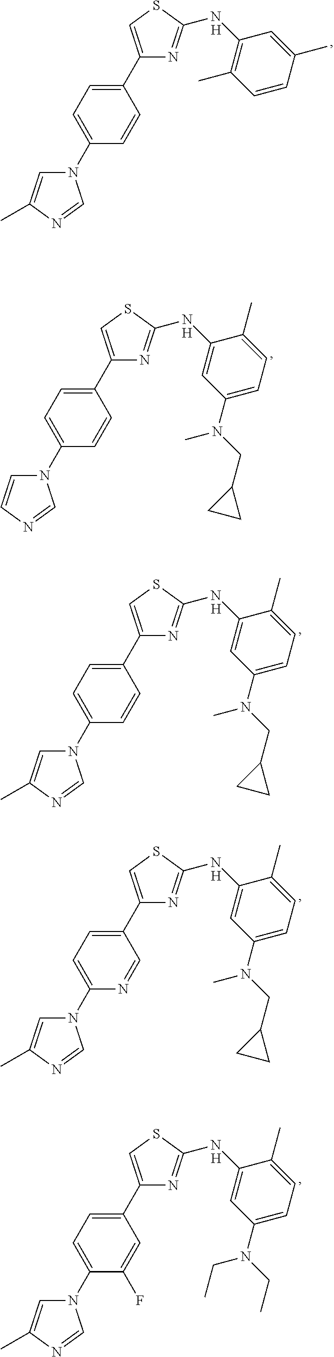

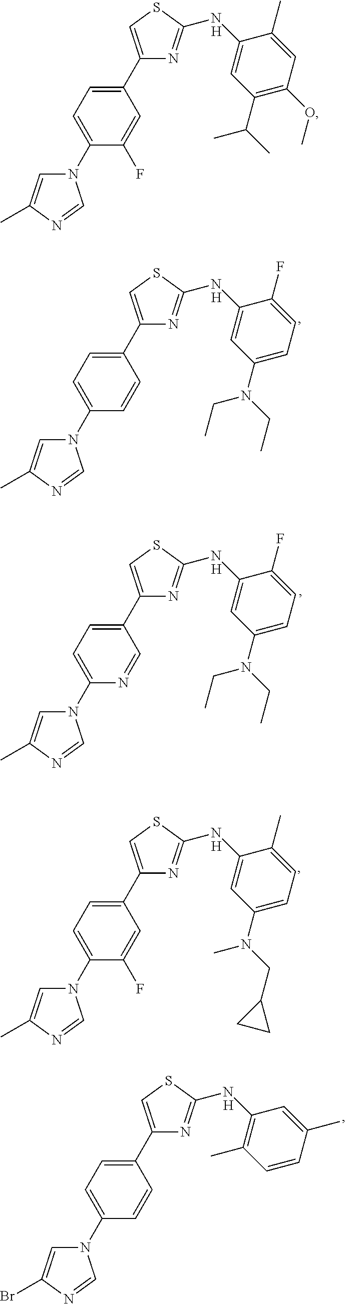

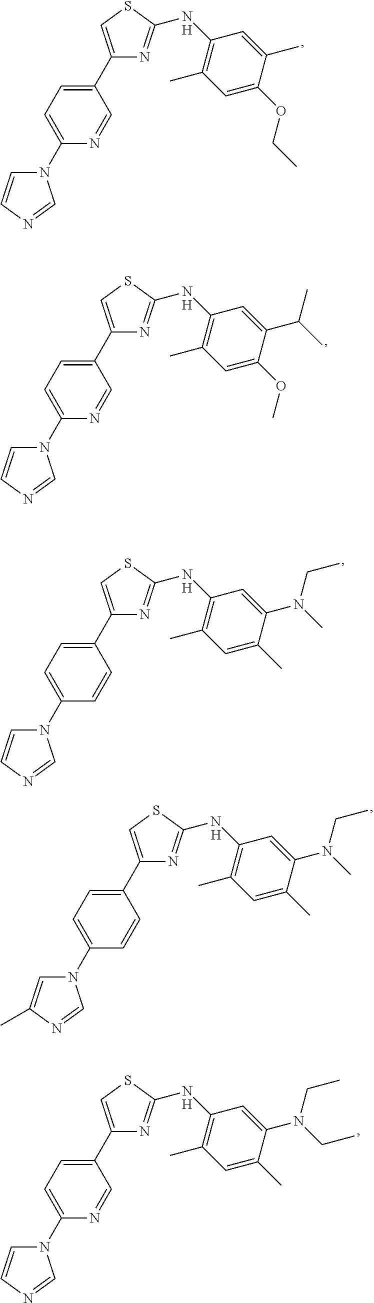

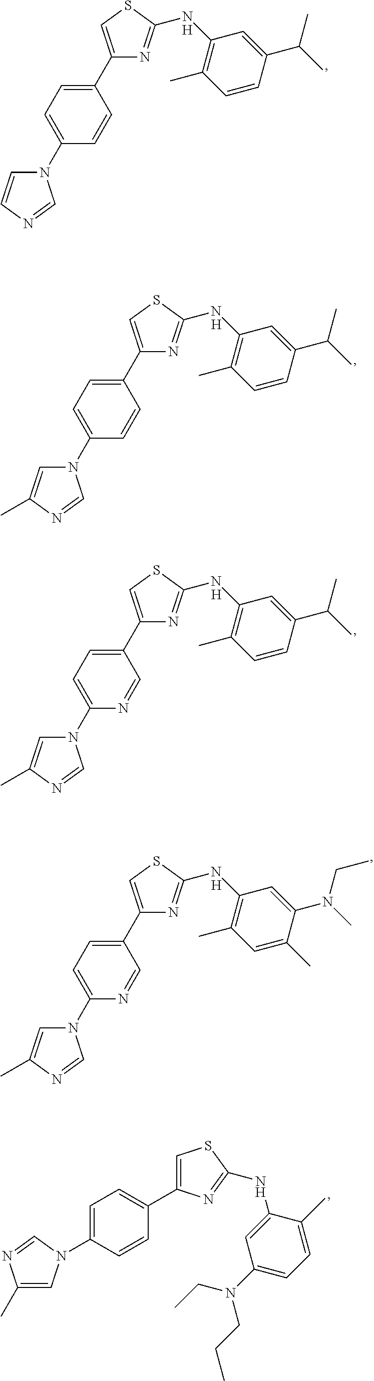

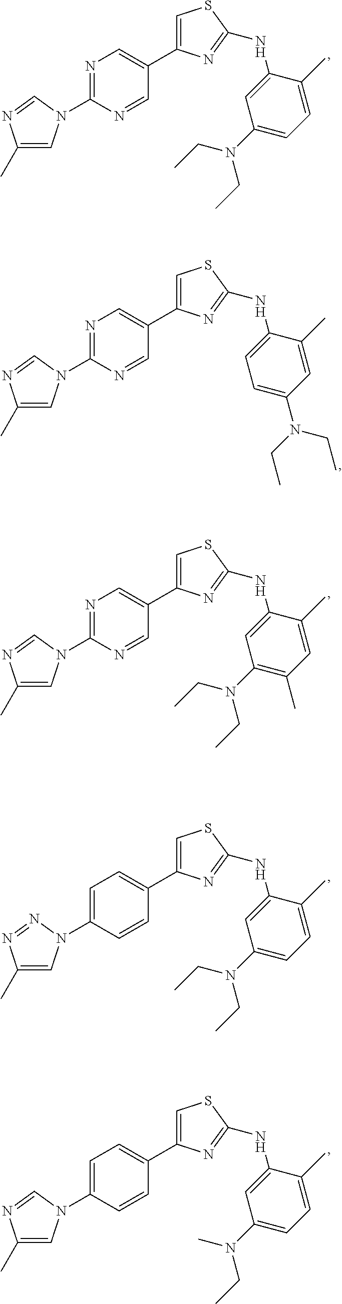

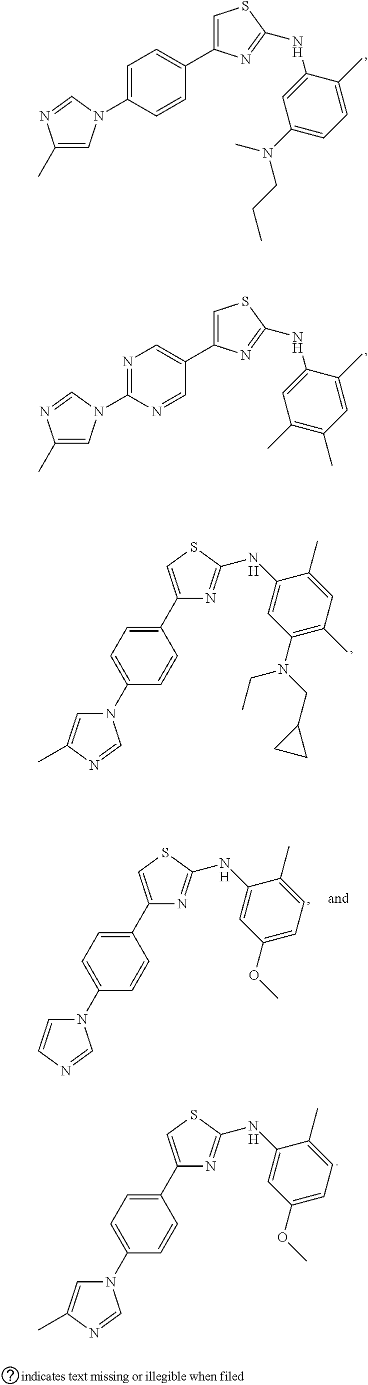

One of skill in the art can readily prepare compounds according to the present invention. See, for example, U.S. Pat. No. 7,244,739, incorporated herein by reference in its entirety.

As used herein, reference to a “finely divided form” of gamma secretase modulators contemplated for use herein embraces particulate matter having a particle size no greater than about 106 μM. In some embodiments, the particle size of finely divided GSMs is no greater than about 53 μM. In some embodiments, the particle size of finely divided GSMs is no greater than 26 μM.

In some embodiments, the particle size distribution of finely divided GSMs according to the present invention falls in the range of 5 μM up to about 109 μM.

Finely divided GSMs according to the present invention can be prepared in a variety of ways, e.g., by ball milling, tumble milling, co-milling, jet milling, high shear mixing, and the like.

As used herein, reference to a “substantially amorphous form” of gamma secretase modulator contemplated for use herein embraces particulate matter lacking significant crystalline structure, i.e., said compound is no greater than 10% crystalline (as observed by X-ray diffraction analysis), in some embodiments no greater than 5% crystalline, in some embodiments no greater than 2% crystalline, and in some embodiments no greater than 1% crystalline.

Substantially amorphous forms of gamma secretase modulators can be prepared in a variety of ways, such as, for example, co-precipitation (in combination with one or more suitable polymeric carrier), spray drying, hot melt extrusion, or the like.

As used herein, “co-precipitation” refers to a process of precipitating two or more components (e.g., a GSM and a polymeric carrier) together from solution. Such precipitation is promoted by non-solvent addition, temperature change, pH change, solvent removal, or the like.

Polymeric carriers contemplated for use herein include polyacrylates and polymethylmethacrylates (e.g., methacrylic acid/ethyl acrylate copolymers, methacrylic acid/methyl methacrylate copolymers, butyl methacrylate/2-dimethylaminoethyl methacrylate copolymers, poly(hydroxyalkyl acrylates), poly(hydroxyalkyl methacrylates, and the like), homopolymers and copolymers of N-vinyl lactams (e.g., polyvinylpyrrolidone, polyvinylpyrrolidone-polyvinylalcohol, and the like), cellulose esters and cellulose ethers (e.g., methylcellulose, ethylcellulose, and the like), hydroxyalkyl celluloses (e.g., hydroxypropyl cellulose, and the like), (hydroxyalkyl)alkyl celluloses (e.g., hydroxypropyl methylcellulose, and the like), cellulose phthalates and succinates (e.g., cellulose acetate phthalate, polyvinyl acetate phthalate, hydroxypropyl cellulose acetate phthalate, hydroxypropylmethyl cellulose acetate phthalate, methylcellulose acetate phthalate, and the like), high molecular weight polyalkylene oxides (e.g., polyethylene oxide, polypropylene oxide, copolymers of ethylene oxide and propylene oxide (also known as poloxamers), and the like), fatty acid PEG esters (e.g., Gelucire), polyacrylamides, vinyl acetate polymers, oligo- and polysaccharides (e.g., carrageenans, galactomannans, xanthan gum, and the like), and the like. A presently preferred polymeric carrier contemplated for use herein is hydroxypropyl methylcellulose acetate succinate.

As used herein, “spray drying” refers to a process whereby a GSM and a suitable polymeric carrier are dissolved in a suitable solvent (e.g., a lower alcohol such as methanol, ethanol, or the like; a ketone such as acetone, methyl ethyl ketone, or the like; etc.), and stirred until a clear solution is obtained, and thereafter the solvent is evaporated therefrom by spraying the clear solution into an evaporative chamber. The resulting spray dried product is then collected for further evaluation.

As used herein, “hot melt extrusion” refers to a process whereby two or more components are mixed using high shear mixing at a controlled temperature. A hot melt extruder typically comprises four primary components:
a motor that controls the rotation of the screws,
the screws (the primary source of shear and movement of the material),
the barrels that house the screws and provide temperature control, and
the die (exit port) that controls the shape and size of the extrudates.
   The feed material (either granular or in powder form) is fed into the extruder feed port at a controlled rate while the extruder screws are rotating. The material is then conveyed forward as a result of the rotation of the screw(s) and the friction of the material against the barrel surface. Depending on the type of extruder, a single screw or a twin screw may be used, operating in either a counter or co-rotating mode. The screws can be appropriately selected to achieve the desired degree of mixing. Typically, the barrels are segmented to enable temperature adjustment in each zone throughout the screw length. The exit port (the die system) controls the shape and size of the final extrudates.

As used herein, reference to gamma secretase modulators in the further presence of one or more excipients therefor contemplate the presence of such excipients as food (e.g., protein, carbohydrate, and the like), polymeric adjuvants (e.g., HPMCAS), a disintegrant (such as, for example, croscarmellose sodium, sodium starch glycolate, or cross-linked povidone), and the like.

The combination of GSM and excipient can be prepared in a variety of ways, e.g., by dissolving in a common solvent (with optional removal of solvent thereafter), by dry blending the two components, by co-grinding the two components together, by co-administering the two components to a subject in need thereof within ±4 hours of one another, and the like.

Thus, in accordance with certain embodiments of the present invention, there are provided formulations comprising:
a GSM,
hydroxypropyl methylcellulose acetate succinate (HPMC-AS),
a disintegrant (e.g., croscarmellose sodium),
a fatty acid PEG ester (e.g., gelucire), and
microcrystalline cellulose (MCC).

In accordance with certain embodiments of the present invention, the above-described formulations provide enhanced transport of one or more GSMs across the blood-brain barrier, relative to prior art formulations.

In accordance with certain embodiments of the present invention, the above-described formulations provide enhanced transport of one or more GSMs from the gut to the bloodstream, relative to prior art formulations.

In accordance with certain embodiments of the present invention, the above-described formulations provide improved GSM compound stability, relative to prior art formulations.

In accordance with certain embodiments of the present invention, the above-described formulations provide less variation in bioavailability of the therapeutically effective GSM compounds.

In some embodiments, a tablet, multiparticulate dosage form, capsule, or granule containing the composition may be coated with an enteric or pH-sensitive layer to facilitate drug composition release in the gastro-intestinal tract distal to the stomach. In some embodiments, the enteric coating or pH-sensitive layer may comprise, but is not limited to, one or more materials selected from the group enteric polymers consisting of cellulose acetate phthalate, cellulose acetate trimellitate, hydroxypropyl methylcellulose acetate succinate, hydroxypropyl methylcellulose phthalate, and polyvinyl acetate phthalate; and anionic polymers based on methacrylic acid and methacrylic acid esters.

In some embodiments, oral administration may be used. Pharmaceutical preparations for oral use can be formulated into conventional oral dosage forms such as capsules, tablets, and liquid preparations such as syrups, elixirs, and concentrated drops. Invention formulations may be combined with solid excipients, optionally grinding a resulting mixture, and processing the mixture of granules, after adding suitable auxiliaries, if desired, to obtain, for example, tablets, coated tablets, hard capsules, soft capsules, solutions (e.g. aqueous, alcoholic, or oily solutions) and the like. Suitable excipients are, in particular, fillers such as sugars, including lactose, glucose, sucrose, mannitol, or sorbitol; cellulose preparations, for example, corn starch, wheat starch, rice starch, potato starch, gelatin, gum tragacanth, methyl cellulose, hydroxypropylmethyl-cellulose, sodium carboxymethylcellulose (CMC), and/or polyvinylpyrrolidone (PVP: povidone); oily excipients, including vegetable and animal oils, such as sunflower oil, olive oil, or codliver oil. The oral dosage formulations may also contain disintegrating agents, such as the cross-linked polyvinylpyrrolidone, agar, or alginic acid, or a salt thereof such as sodium alginate; a lubricant, such as talc or magnesium stearate; a plasticizer, such as glycerol or sorbitol; a sweetening such as sucrose, fructose, lactose, or aspartame; a natural or artificial flavoring agent, such as peppermint, oil of wintergreen, or cherry flavoring; or dye-stuffs or pigments, which may be used for identification or characterization of different doses or combinations. Also provided are dragee cores with suitable coatings. For this purpose, concentrated sugar solutions may be used, which may optionally contain, for example, gum arabic, talc, poly-vinylpyrrolidone, carbopol gel, polyethylene glycol, and/or titanium dioxide, lacquer solutions, and suitable organic solvents or solvent mixtures.

Pharmaceutical preparations that can be used orally include push-fit capsules made of gelatin (“gelcaps”), as well as soft, sealed capsules made of gelatin, and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols.

In some embodiments, injection (parenteral administration) may be used, e.g., intramuscular, intravenous, intraperitoneal, and/or subcutaneous. Invention compositions may be formulated in sterile liquid solutions, preferably in physiologically compatible buffers or solutions, such as saline solution, Hank's solution, or Ringer's solution. Dispersions may also be prepared in non-aqueous solutions, such as glycerol, propylene glycol, ethanol, liquid polyethylene glycols, triacetin, and vegetable oils. Solutions may also contain a preservative, such as methylparaben, propylparaben, chlorobutanol, phenol, sorbic acid, thimerosal, and the like. In addition, the compositions may be formulated in solid form, including, for example, lyophilized forms, and redissolved or suspended prior to use.

In some embodiments, transmucosal, topical or transdermal administration may be used. In such formulations, penetrants appropriate to the barrier to be permeated are used. Such penetrants are generally known in the art, and include, for example, for transmucosal administration, bile salts and fusidic acid derivatives. In addition, detergents may be used to facilitate permeation. Transmucosal administration, for example, may be through nasal sprays or suppositories (rectal or vaginal). Compositions of compounds of Formula I for topical administration may be formulated as oils, creams, lotions, ointments, and the like by choice of appropriate carriers known in the art. Suitable carriers include vegetable or mineral oils, white petrolatum (white soft paraffin), branched chain fats or oils, animal fats and high molecular weight alcohol (greater than C₁₂). In some embodiments, carriers are selected such that the active ingredient is soluble. Emulsifiers, stabilizers, humectants and antioxidants may also be included as well as agents imparting color or fragrance, if desired. Creams for topical application are preferably formulated from a mixture of mineral oil, self-emulsifying beeswax and water in which mixture the active ingredient, dissolved in a small amount of solvent (e.g., an oil), is admixed. Additionally, administration by transdermal means may comprise a transdermal patch or dressing such as a bandage impregnated with an active ingredient and optionally one or more carriers or diluents known in the art. To be administered in the form of a transdermal delivery system, the dosage administration will be continuous rather than intermittent throughout the dosage regimen.

In some embodiments, compounds are administered as inhalants. Invention formulations may be formulated as dry powder or a suitable solution, suspension, or aerosol. Powders and solutions may be formulated with suitable additives known in the art. For example, powders may include a suitable powder base such as lactose or starch, and solutions may comprise propylene glycol, sterile water, ethanol, sodium chloride and other additives, such as acid, alkali and buffer salts. Such solutions or suspensions may be administered by inhaling via spray, pump, atomizer, or nebulizer, and the like. Invention formulations may also be used in combination with other inhaled therapies, for example corticosteroids such as fluticasone proprionate, beclomethasone dipropionate, triamcinolone acetonide, budesonide, and mometasone furoate; beta agonists such as albuterol, salmeterol, and formoterol; anticholinergic agents such as ipratroprium bromide or tiotropium; vasodilators such as treprostinal and iloprost; enzymes such as DNAase; therapeutic proteins; immunoglobulin antibodies; an oligonucleotide, such as single or double stranded DNA or RNA, siRNA; antibiotics such as tobramycin; muscarinic receptor antagonists; leukotriene antagonists; cytokine antagonists; protease inhibitors; cromolyn sodium; nedocril sodium; and sodium cromoglycate.

The amounts of various compounds to be administered can be determined by standard procedures taking into account factors such as the compound activity (in vitro, e.g. the compound IC₅₀ vs. target, or in vivo activity in animal efficacy models), pharmacokinetic results in animal models (e.g. biological half-life or bioavailability), the age, size, and weight of the subject, and the disorder associated with the subject. The importance of these and other factors are well known to those of ordinary skill in the art. Generally, a dose will be in the range of about 0.01 to 50 mg/kg, also about 0.1 to 20 mg/kg of the subject being treated. Multiple doses may be used.

An aspect of the invention is drawn to methods of modulating Aβ levels and methods for treating a disease associated with aberrant Aβ levels using compounds having a structure corresponding to Formula (I). More preferable embodiments herein include methods of modulating Aβ levels and methods for treating a disease associated with aberrant Aβ levels using compounds corresponding to Formulas (II), (III), (IV) or (V).

The phrase “amyloid-beta” or “Aβ” refers to a peptide from a human or other species that (a) results from processing or cleavage of an APP and that is amyloidogenic, (b) is one of the peptide constituents of β-amyloid plaques, (c) is the 43-amino acid sequence of Aβ (amino acid 672-714 of APP770; GenBank Accession No. P05067), (d) is a fragment of a peptide as set forth in (a), (b) or (c), and/or (e) contains one or more additions, deletions or substitutions relative to (a), (b), (c) or (d). Aβ is also referred to in the art as βAP, AβP, or βA4. Aβ peptides derived from proteolysis of APP generally are ˜4.2 kD proteins and are typically 39 to 43 amino acids in length, depending on the carboxy-terminal end-point, which exhibits heterogeneity. However, Aβ peptides containing less than 39 amino acids, e.g., Aβ38, Aβ37, and Aβ34, also may occur.

Aβ peptides can be produced in an amyloidogenic APP processing pathway in which APP is cleaved by β-secretase (BACE) and one or more γ-secretase activities. Aβ peptides include those that begin at position 672 of APP770 and those that begin at position 682 of APP770 (see, for example, GenBank Accession No. P05067). Generally, as used herein, “Aβ” includes any and all Aβ peptides, unless the amino acid residues are specified, such as, for example, 1-43 (Aβ43), 1-42 (Aβ42), 1-40 (Aβ40), 1-39 (Aβ39), 1-38 (Aβ38), 1-37 (Aβ37), 1-34 (Aβ34), 11-43, 11-42, 11-40, 11-39, 11-38, 11-37, 11-34, and others. The various Aβ peptides of differing lengths are referred to herein as “species” of Aβ.

The phrase “amyloid precursor protein” or “APP” refers to a protein that can be proteolytically processed or cleaved by one or more processing or cleavage reactions to produce Aβ. APP includes all isoforms that are generated by alternative splicing, which can be typically distinguished by the number of amino acids in the particular isoform. For example, APP embraces APP695, APP751, and APP770. Other isoforms of APP include, for example, APP714, L-APP752, L-APP733, L-APP696, L-APP677, APP563, and APP365.

APP also includes all isoforms containing mutations found in families with AD and other amyloidosis conditions. For example, these mutations include the Swedish (Lys670Asn, Met671Leu) double mutation; the London mutation (Val717Ile), the Indiana mutation (Val717Leu), Val717Phe, Val717Gly, Ala713Thr, Ala713Val, the Austrian mutation (Thr714Ile), the Iranian mutation (Thr714Ala), the French mutation (Val715Met), the German mutation (Val715Ala), the Florida mutation (Ile716Val), Ile 716Thr, the Australian mutation (Leu723Pro), the Flemish mutation (Ala692Gly), the Dutch mutation (Glu693Gln), the Arctic mutation (Glu693Gly), the Italian mutation (Glu693Lys), and the Iowa mutation (Asp694Asn), and the amyloidsis-Dutch type mutation (Glu693Gln). (All numbering herein is relative to the APP770 form).

The term “APP” further includes proteins containing one or more additions, deletions or substitutions relative to the isoforms described above, and APP proteins from humans and other species. Unless a specific isoform is specified, APP when used herein generally refers to any and all isoforms of APP, with or without mutations, from any species.

The phrase “amyloid precursor protein fragment” refers to any portion of an APP that can be processed or cleaved, by one or more processing or cleavage reactions, to Aβ. Amyloid precursor fragments of APP generally contain either a beta-secretase cleavage site which, when cleaved, generates the N-terminus of Aβ, a gamma-secretase cleavage site which, when cleaved, generates the C-terminus of Aβ or both a beta- and a gamma-secretase cleavage site. Exemplary amyloid precursor fragments include the APP C-terminal fragments designated C99 and C89, as well as portions thereof lacking some or all C-terminal residues that normally reside in the cytosol.

The phrase “source of amyloid precursor protein (APP), amyloid precursor fragment thereof and/or Aβ” refers to any in vivo, ex vivo or in vitro substance containing APP, amyloid precursor fragment thereof and/or Aβ. For example, a “source” can be a live organism (including a human patient, or a laboratory or veterinary animal), a sample therefrom (such as a tissue or body fluid, or extract thereof), a cell (such as a primary cell or cell line, or extract thereof), extracellular medium or matrix or milieu, or isolated protein.

The phrase “modulate” or “modulating” with respect to Aβ level, refers to a detectable increase or decrease in the amount of at least one species of Aβ peptide (such as Aβ43, Aβ42, Aβ40, Aβ39, Aβ38, Aβ37, Aβ34, 11-43, 11-42, 11-40, 11-39, 11-38, 11-37, 11-34, etc.); a detectable increase or decrease in the relative amount of different species of Aβ peptides (such as the ratio of Aβ42 to Aβ40); a detectable increase or decrease in the amount, or relative amount, of Aβ in a particular form (such as monomeric, oligomeric, or fibrillar form; in solution or aggregated in a plaque; in a particular conformation; etc.); and/or a detectable increase or decrease in the amount, or relative amount, of Aβ in a particular location (such as an intracellular, membrane-associated or extracellular location, or in a particular tissue or body fluid). In preferred embodiments, modulation is detectable as a decrease in the level of Aβ42 or Aβ40, or an increase in the level of Aβ37 or Aβ38. Modulation of Aβ level can be evidenced, for example, by an increase or decrease of at least 5%, such as at least 10%, 20%, 30%, 40%, 50%, 75%, 90% or more, of the amount, or relative amount, of an Aβ species, of total Aβ, or of a particular form of Aβ, relative to a reference level. Modulation can be an increase or decrease that is a statistically significant difference relative to the reference level.

The phrase “contacting” refers to bringing into association, either directly or indirectly, two or more substances. Contacting may occur in vivo, ex vivo or in vitro. A source of APP, amyloid precursor fragment thereof and/or Aβ or source of BACE activity, that is a human or other animal can be contacted with a compound, for example, by therapeutic or prophylactic administration of the compound. A source of APP, amyloid precursor fragment thereof and/or Aβ that is a tissue, tissue extract or cell can be contacted with a compound, for example, by introduction of the compound into the culture medium. A source of APP, amyloid precursor fragment thereof and/or Aβ that is a fluid, such as extracellular medium, can be contacted with a compound, for example, by admixing the compound with the fluid.

The phrase “treating” or “treatment” refers to any manner in which one or more of the symptoms of a disease or disorder are ameliorated or otherwise beneficially altered, whether in a permanent or temporary manner, which can be attributed to or associated with administration of the compound or composition herein. The term encompasses any pharmaceutical use, including prophylactic uses in which the development of one or more of the symptoms of a disease or disorder is prevented, delayed or reduced, whether in a permanent or temporary manner, which can be attributed to or associated with administration of the composition. In an embodiment of the invention, treatment encompasses any pharmaceutical use of compounds herein for treating a disease or disorder characterized by altered or aberrant Aβ production, catabolism, processing and/or levels.

The phrase “disease associated with aberrant Aβ levels” refers to any condition characterized by an abnormal amount of at least one species of Aβ peptide (such as Aβ43, Aβ42, Aβ40, Aβ39, Aβ38, Aβ37, Aβ34, 11-43, 11-42, 11-40, 11-39, 11-38, 11-37, 11-34, etc.); by an abnormal relative amount of different species of Aβ peptides (such as the ratio of Aβ42 to Aβ40); by an abnormal amount, or relative amount, of Aβ in a particular form (such as monomeric, oligomeric, or fibrillar form; in solution or aggregated in a plaque; in a particular conformation, etc.); and/or by an abnormal amount, or relative amount, of Aβ in a particular location (such as intracellular, membrane-associated or extracellular location, or in a particular tissue or body fluid). The abnormal amount of one or more Aβ peptides, Aβ forms and/or Aβ can be relative to a condition that is a normal, non-disease state. Diseases and disorders characterized by altered Aβ levels are known in the art and/or described herein, and include, for example, Down syndrome, Parkinson's disease, diffuse Lewy body disease, progressive supranuclear palsy, Hereditary Cerebral Hemorrhage with Amyloidosis-Dutch Type (HCHWA-D), cerebral amyloid angiopathy (CAA), and mild cognitive impairment (MCI). Embodiments of the invention include methods of treating any disease associated with aberrant Aβ levels, such as AD. Compounds of the present invention can be administered to a subject to treat (including to prevent or to ameliorate) conditions associated with altered Aβ production, fibril formation/deposition, degradation and/or clearance, or any altered isoform of Aβ.

Preferably, compounds of the present invention can be used in the treatment of neurological disorders, including but not limited to neurodegenerative conditions and other dementias or traumatic conditions. Exemplary neurological disorders may include diffuse Lewy body disease, Pick's disease, multisystem degeneration (Shy-Drager syndrome), motor neuron diseases including amyotrophic lateral sclerosis, degenerative ataxias, cortical basal degeneration, ALS-Parkinson's-Dementia complex of Guam, subacute sclerosing panencephalitis, Huntington's disease, synucleinopathies, primary progressive aphasia, striatonigral degeneration, Machado-Joseph disease/spinocerebellar ataxia type 3 and olivopontocerebellar degenerations, Gilles De La Tourette's disease, bulbar and pseudobulbar palsy, spinal and spinobulbar muscular atrophy (Kennedy's disease), primary lateral sclerosis, familial spastic paraplegia, Werdnig-Hoffmann disease, Kugelberg-Welander disease, Tay-Sach's disease, Sandhoff disease, familial spastic disease, Wohifart-Kugelberg-Welander disease, spastic paraparesis, progressive multifocal leukoencephalopathy, prion diseases (including Creutzfeldt-Jakob, Gerstmann-Straussler-Scheinker disease, Kuru and fatal familial insomnia), age-related dementia and other conditions with memory loss, such as vascular dementia, diffuse white matter disease (Binswanger's disease), dementia of endocrine or metabolic origin, dementia of head trauma and diffuse brain damage, dementia pugilistica and frontal lobe dementia, cerebral ischemia or infaction including embolic occlusion and thrombotic occlusion as well as intracranial hemorrhage of any type (including, but not limited to, epidural, subdural, subarachnoid and intracerebral), and intracranial and intravertebral lesions (including, but not limited to, contusion, penetration, shear, compression and laceration).

Compounds and compositions of the instant invention may be used to treat or ameliorate a variety of disorders. Compounds and compositions that may be used in therapeutic applications, in one embodiment have reasonably high bioavailability in a target tissue (i.e. brain, for neurodegenerative disorders; particular peripheral organs for other amyloidogenic conditions), and reasonably low toxicity. Those skilled in the art can assess compounds described herein for their pharmaceutical acceptability using standard methods.

For instance, compounds of the instant invention can be used in the treatment of cancer or other diseases characterized by abnormal cellular proliferation, inflammatory disease, bacterial or viral infection, autoimmunue disease, acute pain, muscle pain, neuropathic pain, allergies, neurological disease, dermatological conditions, cardiovascular disease, diabetes, gastrointestinal disorders, depression, endocrine or other disease characterized by abnormal hormonal metabolism, obesity, osteoporosis or other bone disorders, pancreatic disease, epilepsy or seizure disorders, erectile or sexual dysfunction, opthamological disorders or diseases of the eye, cholesterol imbalance, hypertension or hypotension, migraine or headaches, obsessive compulsive disorder, panic disorder, anxiety disorder, post traumatic stress disorder, chemical dependency or addiction, and the like.

Compounds provided herein can also be used to prevent or treat amyloidoses. Amyloidoses include all conditions in which deposition of amyloid in the brain or periphery is a characteristic, including amyloidosis associated with rheumatic diseases, idiopathic diseases, inherited conditions, inflammatory conditions, infectious diseases and malignancies. Amyloidosis disorders include, for example, conditions associated with altered Aβ levels described above (e.g. Alzheimer's disease, Down syndrome, HCHWA-D, cerebral amyloid angiopathy (CAA), and mild cognitive impairment (MCI) etc.), as well as familial amyloid polyneuropathy, familial amyloid cardiomyopathy (Danish type), isolated cardiac amyloid, amyloid angiopathy, systemic senile amyloidosis, familial systemic amyloidosis, light-chain amyloidosis (AL), dialysis-associated amyloidosis, renal amyloidosis, prion-related encephalopathies, diabetes (in which amylin may be deposited in the kidney or pancreas), atrial amyloidosis and pituitary amyloidosis.

Those skilled in the art can determine other diseases and disorders for which administration of a compound or composition described herein can be beneficial.

### Pharmaceutical Compositions

The phrase “pharmaceutically acceptable carrier” refers to any carrier known to those skilled in the art to be suitable for the particular mode of administration. In addition, the compounds may be formulated as the sole pharmaceutically active ingredient in the composition or may be combined with other active ingredients.

The phrase “pharmaceutically acceptable salt” refers to any salt preparation that is appropriate for use in a pharmaceutical application. Pharmaceutically-acceptable salts include amine salts, such as N,N′-dibenzylethylenediamine, chloroprocaine, choline, ammonia, diethanolamine and other hydroxyalkylamines, ethylenediamine, N-methylglucamine, procaine, N-benzylphenethylamine, 1-para-chloro-benzyl-2-pyrrolidin-1′-ylmethylbenzimidazole, diethylamine and other alkylamines, piperazine, tris(hydroxymethyl)aminomethane, and the like; alkali metal salts, such as lithium, potassium, sodium, and the like; alkali earth metal salts, such as barium, calcium, magnesium, and the like; transition metal salts, such as zinc, aluminum, and the like; other metal salts, such as sodium hydrogen phosphate, disodium phosphate, and the like; mineral acids, such as hydrochlorides, sulfates, and the like; and salts of organic acids, such as acetates, lactates, malates, tartrates, citrates, ascorbates, succinates, butyrates, valerates, fumarates, and the like.

The phrase “prodrug” refers to a compound that, upon in vivo administration, is metabolized by one or more steps or processes or otherwise converted to the biologically, pharmaceutically or therapeutically active form of the compound. Prodrugs can be prepared by modifying functional groups present in the compound in such a way that the modifications are cleaved, either in routine manipulation or in vivo, to a compound described herein. For example, prodrugs include compounds of the present invention wherein a hydroxy, amino, or sulfhydryl group is bonded to any group that, when administered to a mammalian subject, can be cleaved to form a free hydroxyl, free amino, or free sulfhydryl group, respectively. Representative prodrugs include, for example, esters, enol ethers, enol esters, acetates, formates, benzoate derivatives, and the like of alcohol and amine functional groups in the compounds of the present invention. By virtue of knowledge of pharmacodynamic processes and drug metabolism in vivo, those of skill in this art, once a pharmaceutically active compound is known, can design prodrugs of the compound (see, e.g., Nogrady (1985) Medicinal Chemistry A Biochemical Approach, Oxford University Press, New York, pages 388-392).

Compositions herein comprise one or more compounds provided herein. The compounds are, in one embodiment, formulated into suitable pharmaceutical preparations such as solutions, suspensions, tablets, dispersible tablets, pills, capsules, powders, sustained release formulations or elixirs, for oral administration or in sterile solutions or suspensions for parenteral administration, as well as transdermal patch preparation and dry powder inhalers. In one embodiment, the compounds described above are formulated into pharmaceutical compositions using techniques and procedures well known in the art (see, e.g., Ansel Introduction to Pharmaceutical Dosage Forms, Fourth Edition 1985, 126).

In the compositions, effective concentrations of one or more compounds or pharmaceutically acceptable derivatives thereof is (are) mixed with a suitable pharmaceutical carrier. The compounds may be derivatized as the corresponding salts, esters, enol ethers or esters, acetals, ketals, orthoesters, hemiacetals, hemiketals, acids, bases, solvates, hydrates or prodrugs prior to formulation, as described above. The concentrations of the compounds in the compositions are effective for delivery of an amount, upon administration, that treats, prevents, or ameliorates one or more of the symptoms of diseases or disorders to be treated.

In one embodiment, the compositions are formulated for single dosage administration. To formulate a composition, the weight fraction of compound is dissolved, suspended, dispersed or otherwise mixed in a selected carrier at an effective concentration such that the treated condition is relieved, prevented, or one or more symptoms are ameliorated.

The active compound is included in the pharmaceutically acceptable carrier in an amount sufficient to exert a therapeutically useful effect in the absence of undesirable side effects on the patient treated. The therapeutically effective concentration may be determined empirically by testing the compounds in in vitro and in vivo systems described herein and in PCT publication WO 04/018997, and then extrapolated therefrom for dosages for humans.

The concentration of active compound in the pharmaceutical composition will depend on absorption, inactivation and excretion rates of the active compound, the physicochemical characteristics of the compound, the dosage schedule, and amount administered as well as other factors known to those of skill in the art.

In one embodiment, a therapeutically effective dosage should produce a serum concentration of active ingredient of from about 0.1 ng/ml to about 50-100 μg/ml. The pharmaceutical compositions, in another embodiment, should provide a dosage of from about 0.001 mg to about 2000 mg of compound per kilogram of body weight per day. Pharmaceutical dosage unit forms are prepared to provide from about 0.01 mg, 0.1 mg or 1 mg to about 500 mg, 1000 mg or 2000 mg, and in one embodiment from about 10 mg to about 500 mg of the active ingredient or a combination of essential ingredients per dosage unit form.

The active ingredient may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

In instances in which the compounds exhibit insufficient solubility, methods for solubilizing compounds may be used. Such methods are known to those of skill in this art, and include, but are not limited to, using cosolvents, such as dimethylsulfoxide (DMSO), using surfactants, such as TWEEN®, or dissolution in aqueous sodium bicarbonate. Derivatives of the compounds, such as prodrugs of the compounds may also be used in formulating effective pharmaceutical compositions.

Upon mixing or addition of the compound(s), the resulting mixture may be a solution, suspension, emulsion or the like. The form of the resulting mixture depends upon a number of factors, including the intended mode of administration and the solubility of the compound in the selected carrier or vehicle. The effective concentration is sufficient for ameliorating the symptoms of the disease, disorder or condition treated and may be empirically determined.

The pharmaceutical compositions are provided for administration to humans and animals in unit dosage forms, such as tablets, capsules, pills, powders, granules, sterile parenteral solutions or suspensions, and oral solutions or suspensions, and oil-water emulsions containing suitable quantities of the compounds or pharmaceutically acceptable derivatives thereof. The pharmaceutically therapeutically active compounds and derivatives thereof are, in one embodiment, formulated and administered in unit-dosage forms or multiple-dosage forms. Unit-dose forms as used herein refers to physically discrete units suitable for human and animal subjects and packaged individually as is known in the art. Each unit-dose contains a predetermined quantity of the therapeutically active compound sufficient to produce the desired therapeutic effect, in association with the required pharmaceutical carrier, vehicle or diluent. Examples of unit-dose forms include ampoules and syringes and individually packaged tablets or capsules. Unit-dose forms may be administered in fractions or multiples thereof. A multiple-dose form is a plurality of identical unit-dosage forms packaged in a single container to be administered in segregated unit-dose form. Examples of multiple-dose forms include vials, bottles of tablets or capsules or bottles of pints or gallons. Hence, multiple dose form is a multiple of unit-doses which are not segregated in packaging.

Liquid pharmaceutically administrable compositions can, for example, be prepared by dissolving, dispersing, or otherwise mixing an active compound as defined above and optional pharmaceutical adjuvants in a carrier, such as, for example, water, saline, aqueous dextrose, glycerol, glycols, ethanol, and the like, to thereby form a solution or suspension. If desired, the pharmaceutical composition to be administered may also contain minor amounts of nontoxic auxiliary substances such as wetting agents, emulsifying agents, solubilizing agents, pH buffering agents and the like, for example, acetate, sodium citrate, cyclodextrine derivatives, sorbitan monolaurate, triethanolamine sodium acetate, triethanolamine oleate, and other such agents.

Actual methods of preparing such dosage forms are known, or will be apparent, to those skilled in this art; for example, see Remington's Pharmaceutical Sciences, Mack Publishing Company, Easton, Pa., 15th Edition, 1975.

Dosage forms or compositions containing active ingredient in the range of 0.005% to 100% (wt %) with the balance made up from non-toxic carrier may be prepared. Methods for preparation of these compositions are known to those skilled in the art. The contemplated compositions may contain 0.001%-100% (wt %) active ingredient, in one embodiment 0.1-95% (wt %), in another embodiment 75-85% (wt %).

### Compositions for Oral Administration

Oral pharmaceutical dosage forms are either solid, gel or liquid. The solid dosage forms are tablets, capsules, granules, and bulk powders. Types of oral tablets include compressed, chewable lozenges and tablets which may be enteric-coated, sugar-coated or film-coated. Capsules may be hard or soft gelatin capsules, while granules and powders may be provided in non-effervescent or effervescent form with the combination of other ingredients known to those skilled in the art.

Solid Compositions for Oral Administration

In certain embodiments, the formulations are solid dosage forms, in one embodiment, capsules or tablets. The tablets, pills, capsules, troches and the like can contain one or more of the following ingredients, or compounds of a similar nature: a binder; a lubricant; a diluent; a glidant; a disintegrating agent; a coloring agent; a sweetening agent; a flavoring agent; a wetting agent; an emetic coating; and a film coating. Examples of binders include microcrystalline cellulose, gum tragacanth, glucose solution, acacia mucilage, gelatin solution, molasses, polvinylpyrrolidine, povidone, crospovidones, sucrose and starch paste. Lubricants include talc, starch, magnesium or calcium stearate, lycopodium and stearic acid. Diluents include, for example, lactose, sucrose, starch, kaolin, salt, mannitol and dicalcium phosphate. Glidants include, but are not limited to, colloidal silicon dioxide. Disintegrating agents include crosscarmellose sodium, sodium starch glycolate, alginic acid, corn starch, potato starch, bentonite, methylcellulose, agar and carboxymethylcellulose. Coloring agents include, for example, any of the approved certified water soluble FD and C dyes, mixtures thereof; and water insoluble FD and C dyes suspended on alumina hydrate. Sweetening agents include sucrose, lactose, mannitol and artificial sweetening agents such as saccharin, and any number of spray dried flavors. Flavoring agents include natural flavors extracted from plants such as fruits and synthetic blends of compounds which produce a pleasant sensation, such as, but not limited to peppermint and methyl salicylate. Wetting agents include propylene glycol monostearate, sorbitan monooleate, diethylene glycol monolaurate and polyoxyethylene laural ether. Emetic-coatings include fatty acids, fats, waxes, shellac, ammoniated shellac and cellulose acetate phthalates. Film coatings include hydroxyethylcellulose, sodium carboxymethylcellulose, polyethylene glycol 4000 and cellulose acetate phthalate.

The compound, or pharmaceutically acceptable derivative thereof, could be provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

When the dosage unit form is a capsule, it can contain, in addition to material of the above type, a liquid carrier such as a fatty oil. In addition, dosage unit forms can contain various other materials which modify the physical form of the dosage unit, for example, coatings of sugar and other enteric agents. The compounds can also be administered as a component of an elixir, suspension, syrup, wafer, sprinkle, chewing gum or the like. A syrup may contain, in addition to the active compounds, sucrose as a sweetening agent and certain preservatives, dyes and colorings and flavors.

The active materials can also be mixed with other active materials which do not impair the desired action, or with materials that supplement the desired action, such as antacids, H2 blockers, and diuretics. The active ingredient is a compound or pharmaceutically acceptable derivative thereof as described herein. Higher concentrations, up to about 98% by weight of the active ingredient may be included.

In all embodiments, tablets and capsules formulations may be coated as known by those of skill in the art in order to modify or sustain dissolution of the active ingredient. Thus, for example, they may be coated with a conventional enterically digestible coating, such as phenylsalicylate, waxes and cellulose acetate phthalate.

Liquid Compositions for Oral Administration

Liquid oral dosage forms include aqueous solutions, emulsions, suspensions, solutions and/or suspensions reconstituted from non-effervescent granules and effervescent preparations reconstituted from effervescent granules. Aqueous solutions include, for example, elixirs and syrups. Emulsions are either oil-in-water or water-in-oil.

Elixirs are clear, sweetened, hydroalcoholic preparations. Pharmaceutically acceptable carriers used in elixirs include solvents. Syrups are concentrated aqueous solutions of a sugar, for example, sucrose, and may contain a preservative. An emulsion is a two-phase system in which one liquid is dispersed in the form of small globules throughout another liquid. Pharmaceutically acceptable carriers used in emulsions are non-aqueous liquids, emulsifying agents and preservatives. Suspensions use pharmaceutically acceptable suspending agents and preservatives. Pharmaceutically acceptable substances used in non-effervescent granules, to be reconstituted into a liquid oral dosage form, include diluents, sweeteners and wetting agents. Pharmaceutically acceptable substances used in effervescent granules, to be reconstituted into a liquid oral dosage form, include organic acids and a source of carbon dioxide. Coloring and flavoring agents are used in all of the above dosage forms.

Solvents include glycerin, sorbitol, ethyl alcohol and syrup. Examples of preservatives include glycerin, methyl and propylparaben, benzoic acid, sodium benzoate and alcohol. Examples of non-aqueous liquids utilized in emulsions include mineral oil and cottonseed oil. Examples of emulsifying agents include gelatin, acacia, tragacanth, bentonite, and surfactants such as polyoxyethylene sorbitan monooleate. Suspending agents include sodium carboxymethylcellulose, pectin, tragacanth, Veegum and acacia. Sweetening agents include sucrose, syrups, glycerin and artificial sweetening agents such as saccharin. Wetting agents include propylene glycol monostearate, sorbitan monooleate, diethylene glycol monolaurate and polyoxyethylene lauryl ether. Organic acids include citric and tartaric acid. Sources of carbon dioxide include sodium bicarbonate and sodium carbonate. Coloring agents include any of the approved certified water soluble FD and C dyes, and mixtures thereof. Flavoring agents include natural flavors extracted from plants such fruits, and synthetic blends of compounds which produce a pleasant taste sensation.

For a solid dosage form, the solution or suspension, in for example propylene carbonate, vegetable oils or triglycerides, is in one embodiment encapsulated in a gelatin capsule. Such solutions, and the preparation and encapsulation thereof, are disclosed in U.S. Pat. Nos. 4,328,245; 4,409,239; and 4,410,545. For a liquid dosage form, the solution, e.g., for example, in a polyethylene glycol, may be diluted with a sufficient quantity of a pharmaceutically acceptable liquid carrier, e.g., water, to be easily measured for administration.

Alternatively, liquid or semi-solid oral formulations may be prepared by dissolving or dispersing the active compound or salt in vegetable oils, glycols, triglycerides, propylene glycol esters (e.g., propylene carbonate) and other such carriers, and encapsulating these solutions or suspensions in hard or soft gelatin capsule shells. Other useful formulations include those set forth in U.S. Pat. Nos. RE28,819 and 4,358,603. Briefly, such formulations include, but are not limited to, those containing a compound provided herein, a dialkylated mono- or polyalkylene glycol, including, but not limited to, 1,2-dimethoxymethane, diglyme, triglyme, tetraglyme, polyethylene glycol-350-dimethyl ether, polyethylene glycol-550-dimethyl ether, polyethylene glycol-750-dimethyl ether wherein 350, 550 and 750 refer to the approximate average molecular weight of the polyethylene glycol, and one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, thiodipropionic acid and its esters, and dithiocarbamates.

Other formulations include, but are not limited to, aqueous alcoholic solutions including a pharmaceutically acceptable acetal. Alcohols used in these formulations are any pharmaceutically acceptable water-miscible solvents having one or more hydroxyl groups, including, but not limited to, propylene glycol and ethanol. Acetals include, but are not limited to, di(lower alkyl) acetals of lower alkyl aldehydes such as acetaldehyde diethyl acetal.

Injectables, Solutions and Emulsions

Parenteral administration, in one embodiment characterized by injection, either subcutaneously, intramuscularly or intravenously is also contemplated herein. Injectables can be prepared in conventional forms, either as liquid solutions or suspensions, solid forms suitable for solution or suspension in liquid prior to injection, or as emulsions. The injectables, solutions and emulsions also contain one or more excipients. Suitable excipients are, for example, water, saline, dextrose, glycerol or ethanol. In addition, if desired, the pharmaceutical compositions to be administered may also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins.

Implantation of a slow-release or sustained-release system, such that a constant level of dosage is maintained (see, e.g. U.S. Pat. No. 3,710,795) is also contemplated herein. Briefly, a compound provided herein is dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The compound diffuses through the outer polymeric membrane in a release rate controlling step. The percentage of active compound contained in such parenteral compositions is highly dependent on the specific nature thereof, as well as the activity of the compound and the needs of the subject.

Parenteral administration of the compositions includes intravenous, subcutaneous and intramuscular administrations. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations must be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN® 80). A sequestering or chelating agent of metal ions include EDTA. Pharmaceutical carriers also include ethyl alcohol, polyethylene glycol and propylene glycol for water miscible vehicles; and sodium hydroxide, hydrochloric acid, citric acid or lactic acid for pH adjustment.

The concentration of the pharmaceutically active compound is adjusted so that an injection provides an effective amount to produce the desired pharmacological effect. The exact dose depends on the age, weight and condition of the patient or animal as is known in the art.

The unit-dose parenteral preparations are packaged in an ampoule, a vial or a syringe with a needle. All preparations for parenteral administration must be sterile, as is known and practiced in the art.

Illustratively, intravenous or intraarterial infusion of a sterile aqueous solution containing an active compound is an effective mode of administration. Another embodiment is a sterile aqueous or oily solution or suspension containing an active material injected as necessary to produce the desired pharmacological effect.

Injectables are designed for local and systemic administration. In one embodiment, a therapeutically effective dosage is formulated to contain a concentration of at least about 0.1% w/w up to about 90% w/w or more, in certain embodiments more than 1% w/w of the active compound to the treated tissue(s).

The compound may be suspended in micronized or other suitable form or may be derivatized to produce a more soluble active product or to produce a prodrug. The form of the resulting mixture depends upon a number of factors, including the intended mode of administration and the solubility of the compound in the selected carrier or vehicle. The effective concentration is sufficient for ameliorating the symptoms of the condition and may be empirically determined.

Lyophilized Powders

Of interest herein are also lyophilized powders, which can be reconstituted for administration as solutions, emulsions and other mixtures. They may also be reconstituted and formulated as solids or gels.

The sterile, lyophilized powder is prepared by dissolving a compound provided herein, or a pharmaceutically acceptable derivative thereof, in a suitable solvent. The solvent may contain an excipient which improves the stability or other pharmacological component of the powder or reconstituted solution, prepared from the powder. Excipients that may be used include, but are not limited to, dextrose, sorbital, fructose, corn syrup, xylitol, glycerin, glucose, sucrose or other suitable agent. The solvent may also contain a buffer, such as citrate, sodium or potassium phosphate or other such buffer known to those of skill in the art at, in one embodiment, about neutral pH. Subsequent sterile filtration of the solution followed by lyophilization under standard conditions known to those of skill in the art provides the desired formulation. In one embodiment, the resulting solution will be apportioned into vials for lyophilization. Each vial will contain a single dosage or multiple dosages of the compound. The lyophilized powder can be stored under appropriate conditions, such as at about 4° C. to room temperature.

Reconstitution of this lyophilized powder with water for injection provides a formulation for use in parenteral administration. For reconstitution, the lyophilized powder is added to sterile water or other suitable carrier. The precise amount depends upon the selected compound. Such amount can be empirically determined.

Topical Administration

Topical mixtures are prepared as described for the local and systemic administration. The resulting mixture may be a solution, suspension, emulsions or the like and are formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

The compounds or pharmaceutically acceptable derivatives thereof may be formulated as aerosols for topical application, such as by inhalation (see, e.g., U.S. Pat. Nos. 4,044,126, 4,414,209, and 4,364,923, which describe aerosols for delivery of a steroid useful for treatment of inflammatory diseases, particularly asthma). These formulations for administration to the respiratory tract can be in the form of an aerosol or solution for a nebulizer, or as a microfine powder for insufflation, alone or in combination with an inert carrier such as lactose. In such a case, the particles of the formulation will, in one embodiment, have diameters of less than 50 microns, in one embodiment less than 10 microns.

The compounds may be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the active compound alone or in combination with other pharmaceutically acceptable excipients can also be administered.

These solutions, particularly those intended for ophthalmic use, may be formulated as 0.01%-10% (vol %) isotonic solutions, pH about 5-7, with appropriate salts.

### Compositions for Other Routes of Administration

Other routes of administration, such as transdermal patches, including iontophoretic and electrophoretic devices, and rectal administration, are also contemplated herein.

Transdermal patches, including iotophoretic and electrophoretic devices, are well known to those of skill in the art. For example, such patches are disclosed in U.S. Pat. Nos. 6,267,983, 6,261,595, 6,256,533, 6,167,301, 6,024,975, 6,010715, 5,985,317, 5,983,134, 5,948,433, and 5,860,957.

For example, pharmaceutical dosage forms for rectal administration are rectal suppositories, capsules and tablets for systemic effect. Rectal suppositories are used herein mean solid bodies for insertion into the rectum which melt or soften at body temperature releasing one or more pharmacologically or therapeutically active ingredients. Pharmaceutically acceptable substances utilized in rectal suppositories are bases or vehicles and agents to raise the melting point. Examples of bases include cocoa butter (theobroma oil), glycerin-gelatin, carbowax (polyoxyethylene glycol) and appropriate mixtures of mono-, di- and triglycerides of fatty acids. Combinations of the various bases may be used. Agents to raise the melting point of suppositories include spermaceti and wax. Rectal suppositories may be prepared either by the compressed method or by molding. The weight of a rectal suppository, in one embodiment, is about 2 to 3 gm.

Tablets and capsules for rectal administration are manufactured using the same pharmaceutically acceptable substance and by the same methods as for formulations for oral administration.

Targeted Formulations

The compounds provided herein, or pharmaceutically acceptable derivatives thereof, may also be formulated to be targeted to a particular tissue, receptor, or other area of the body of the subject to be treated. Many such targeting methods are well known to those of skill in the art. All such targeting methods are contemplated herein for use in the instant compositions. For non-limiting examples of targeting methods, see, e.g., U.S. Pat. Nos. 6,316,652, 6,274,552, 6,271,359, 6,253,872, 6,139,865, 6,131,570, 6,120,751, 6,071,495, 6,060,082, 6,048,736, 6,039,975, 6,004,534, 5,985,307, 5,972,366, 5,900,252, 5,840,674, 5,759,542 and 5,709,874.

In one embodiment, liposomal suspensions, including tissue-targeted liposomes, such as tumor-targeted liposomes, may also be suitable as pharmaceutically acceptable carriers. These may be prepared according to methods known to those skilled in the art. For example, liposome formulations may be prepared as described in U.S. Pat. No. 4,522,811. Briefly, liposomes such as multilamellar vesicles (MLV's) may be formed by drying down egg phosphatidyl choline and brain phosphatidyl serine (7:3 molar ratio) on the inside of a flask. A solution of a compound provided herein in phosphate buffered saline lacking divalent cations (PBS) is added and the flask shaken until the lipid film is dispersed. The resulting vesicles are washed to remove unencapsulated compound, pelleted by centrifugation, and then resuspended in PBS.

The following examples are provided to describe the invention in greater detail. The examples are intended illustrate, not to limit, the invention.

### Example 1

### General Experimental Procedures

Test Article Preparation

Spray Dried Dispersion Product: A 7571 g spray solution containing 5.6% (w/w) NGP555, 5.6% (w/w) HPMC-AS, and 88.8% (w/w) methanol is prepared by adding 424 g of NGP555 API to 6723 g of methanol. The solution is stirred using a paddle mixer until all API has dissolved. Once all API has dissolved, 424 g of HPMC-AS is added to the solution and mixed using a paddle mixer overnight.

The solution is then spray dried using the Buchi B-290 parameters listed in the following table as a guideline. All operating parameters are recorded.

TABLE
Buchi B-290 Parameters

| Parameter | Description |
|---|---|
| System Setup | Closed Loop |
| Glassware Setup | Standard |
| Aspirator Rate (%) | 85 |
| Inlet Temperature Setting | 130° C. |
| Target Inlet Temperature | ~130° C. |
| Target Exhaust Temperature | ~65° C. |
| Atomizing Air Pressure (mm) | 45 |
| Pump Speed (g/min) | 16.5 (~60% Setting) |
| Needle Purge | 2 |

Once all solution has been sprayed, an aliquot of approximately 1 g is removed, stored in a glass vial, and purged with N₂. The remaining material from the main collection vessel is transferred and stored in bulk within a glass vessel and purged with N₂.

A portion of the 1 g dried material aliquot is analyzed by TGA and DSC prior to formulation preparation.

Approximately 100 mg of the dried material aliquot is subjected to XRPD testing.

A total of four spray solutions are prepared, spray dried, and analyzed as noted above. Only one spray solution is sprayed per working day; the resulting dried material is stored in a glass bottle at 5° C. and purged with N₂. Once all spray solutions have been sprayed and the resulting dried material analyzed, the bulk materials are combined. An aliquot of approximately 10 g combined, dried material is removed, stored in a glass vial, and purged with N₂.

Active Blend Preparation

The yield of total spray-dried dispersion product from all four spray solution preparations is calculated and the amounts of MCC and croscarmellose sodium are determined using the % composition values listed in following Table. The dispersion product and croscarmellose sodium are sandwiched between the MCC within the blending vessel prior to blending.

TABLE
Active Formulation Blend Composition

| Material | % Composition |
|---|---|
| Dispersion Product | 40.0 |
| MCC | 55.0 |
| Croscarmellose Sodium | 5.0 |

The contents of the blending vessel are mixed on the Turbula mixer for 5 minutes at 32 RPM, midstream sieved through a 250 μm sieve screen into a collection pan, then placed back in the same blending vessel and mixed for an additional 5 minutes at 32 RPM.

Placebo Formulation

A 4500 g formulation blend is prepared by adding the appropriate amount of each excipient listed in the following Table to a blending vessel. The contents of the blending vessel are mixed on the Turbula mixer for 10 minutes at 32 RPM.

TABLE
Placebo Formulation Blend Composition

| Material | % Composition | Amount |
|---|---|---|
| HPMC-AS | 20.0 | 900 g |
| MCC | 75.0 | 3375 g |
| Croscarmellose Sodium | 5.0 | 225 g |

Standard

NGP555 API, Cedarburg-Hauser, lot 1722-34, is used as a standard for sample quantitation. This material is not an official reference standard, but is equivalent to the material used to prepare the NGP555 active formulation product. This material is stored at 5° C./Ambient RH.

Spray Dried Dispersion Preparations

The following spray dried dispersions were prepared for dissolution evaluations:

### 1) NGP555 Alone

2) NGP555:HPMC-AS 1:1 (current GLP formulation)

### 3) NGP555:HPMC-AS 4:1

### 4) NGP555:HPMC 1:1

### 5) NGP555:HPMC 4:1

### 6) NGP555:PVP 1:1

### 7) NGP555:PVP 4:1

### 8) NGP555:PEG4000 1:1

To prepare each spray dried dispersion, the components are weighed and added to 39.5 g of MeOH (50 mL) at a total solids content of 5.95% (w/w). The solutions containing HPMC-AS and HPMC are stirred overnight. The solutions containing PVP and PEG do not need to be stirred overnight since the solutions become clear after ˜10 minutes of mixing. Each solution is sprayed using the spray drying parameters listed in the following Table.

TABLE
Spray Drying Parameters

| Parameter | Description |
|---|---|
| System Setup | Closed Loop |
| Glassware Setup | High Efficiency |
| Aspirator Rate (%) | 85 |
| Inlet Temperature Setting | 130° C. |
| Target Inlet Temperature | ~130° C. |
| Target Exhaust Temperature | ~50° C. |
| Atomizing Air Pressure (mm) | 40 |
| Pump Speed (g/min) | 16 (63% Setting) |
| Needle Purge | 2 |

Once each spray solution has been spray dried, the resulting dispersion product is transferred to a glass vial, purged with N₂, and stored at 5° C. The spray dried dispersion containing NGP555:PEG4000 at 1:1 is not able to be recovered due to melting of the product occurring in the spray dryer. An additional spray solution at NGP555:PEG4000 4:1 is prepared and spray dried which results in a solid product. The recovery for each dispersion product is listed in the following Table.

TABLE
Dispersion Product Recovery

| Dispersion Product | Recovery Wt, g | Target Wt, g | % Recovery |
|---|---|---|---|
| NGP555 Alone | 1.534 | 2.500 | 61.4 |
| NGP555:HPMC-AS 1:1 | 1.785 | | 71.4 |
| NGP555:HPMC-AS 4:1 | 1.616 | | 64.6 |
| NGP555:HPMC 1:1 | 1.702 | | 68.1 |
| NGP555:HPMC 4:1 | 1.826 | | 73.0 |
| NGP555:PVP 1:1 | 1.731 | | 69.2 |
| NGP555:PVP 4:1 | 1.870 | | 74.8 |
| NGP555:PEG4000 1:1 | N/A | | N/A |
| NGP555:PEG4000 4:1 | 1.719 | | 68.8 |

Each dispersion product is tested for residual solvents by TGA. The results from the TGA are listed in the following Table.

TABLE
TGA Results

| Dispersion Product | % Total |
|---|---|
| NGP555 Alone | 2.887 |
| NGP555:HPMC-AS 1:1 | 3.093 |
| NGP555:HPMC-AS 4:1 | 2.788 |
| NGP555:HPMC 1:1 | 5.378 |
| NGP555:HPMC 4:1 | 3.785 |
| NGP555:PVP 1:1 | 3.387 |
| NGP555:PVP 4:1 | 3.773 |
| NGP555:PEG4000 4:1 | 3.323 |

The dispersion products are analyzed by DSC.

Formulation Blend and Tablet Preparations

All blends are assumed to have 3% residual solvents based on historical data. The HCl salt content, ROI, and purity are not corrected for in the formulation blend, but instead are calculated in the theoretical concentration for the assay/RS and dissolution sample preparations (see below).

A 1 g formulation blend is prepared for each dispersion product using an excipient blend representative of the current GLP formulation with a target tablet weight of 250 mg (See the following Table).

TABLE
GLP Formulation Composition

| Component | % of Blend | Amt/Tablet, mg |
|---|---|---|
| Dispersion Product | 41.24 | 103.1 |
| MCC | 53.76 | 134.4 |
| Cros. Sodium | 5.00 | 12.5 |

The amount of croscarmellose sodium is kept constant in each formulation at 5% (w/w), but the amount of MCC is adjusted based on the amount of dispersion product added to each formulation. Blends are prepared in glass vials and hand mixed for 1 minute prior to tableting.

For each formulation blend, 2 tablets are pressed at ˜250 mg fill weight for a target dosage strength of 50 mg (uncorrected for HCl salt content, ROI, and purity). A formulation blend aliquot is weighed and added to a single station tablet press fit with 5/16″ tooling. Each blend aliquot is pressed to ˜800 psi and the resulting tablets are stored in glass vials to be used for dissolution evaluations.

### Example 2

### Assay/RS Analysis

Each dispersion product is tested by HPLC. A portion of each dispersion product is prepared in MeOH to target a sample concentration of 500 μg/mL and analyzed against a reference standard also prepared at 500 μg/mL using the same lot of API (Cedarburg-Hauser, lot 1722-34). The potency and purity results for each dispersion product are listed in the following Table.

TABLE
HPLC Potency and Purity Results

| Dispersion Product | % Potency | Purity (area %) |
|---|---|---|
| NGP555 Alone | 99.6 | 99.6 |
| NGP555:HPMC-AS 1:1 | 101.7 | 99.7 |
| NGP555:HPMC-AS 4:1 | 99.1 | 99.7 |
| NGP555:HPMC 1:1 | 104.0 | 99.6 |
| NGP555:HPMC 4:1 | 101.6 | 99.7 |
| NGP555:PVP 1:1 | 100.5 | 99.5 |
| NGP555:PVP 4:1 | 98.9 | 99.7 |
| NGP555:PEG4000 4:1 | 101.8 | 99.7 |

### Dissolution Evaluation

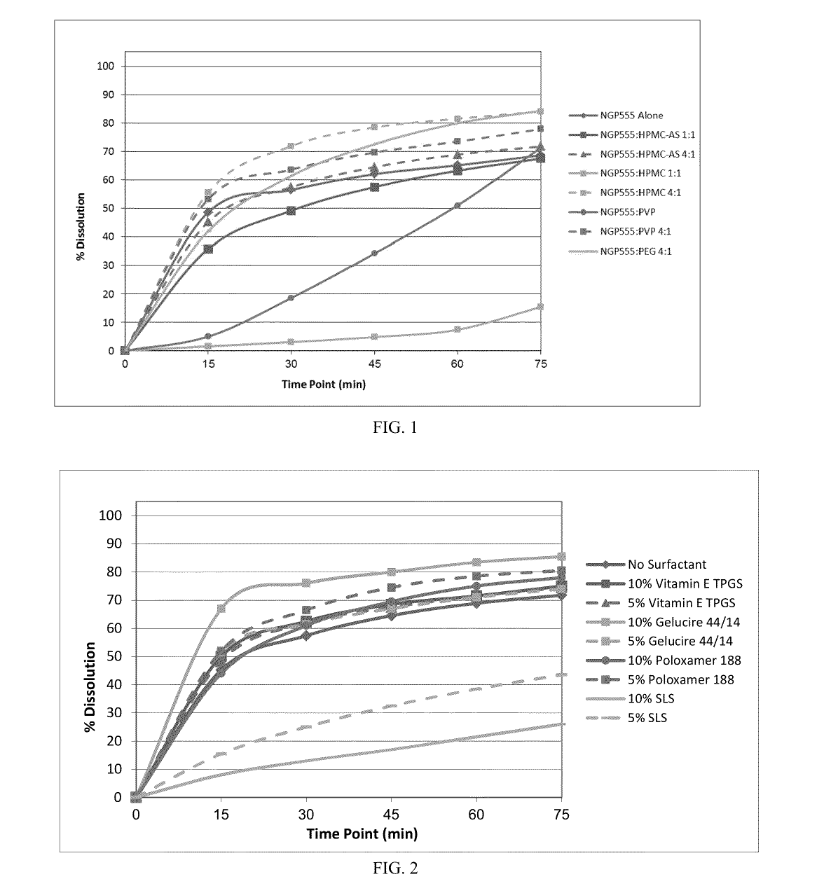

For each formulation, the 2 tablets prepared as described above are tested by dissolution according to the following dissolution parameters: 100 RPM paddle speed and 37.0° C. using USP apparatus 2 (Paddles) in 900 mL of 0.1N HCl. A 3 mL aliquot is removed at 15, 30, 45, 60, and 75 minutes from each vessel and filtered through a 0.45 μm nylon filter into an HPLC vial, discarding the first 2 mL of filtrate. After 60 minutes, the paddle speed is increased to 200 RPM. Samples are quantitated based on a standard prepared at ˜50 μg/mL (the 500 μg/mL standard above diluted 1:10 in 0.1N HCl). The average dissolution is calculated for the duplicate samples of each formulation. The dissolution profile for each formulation is shown in FIG. 1.

In conclusion, it has been observed that:
The tablets containing the API:HPMC 1:1 dispersion product do not disintegrate throughout the dissolution run.
The tablets containing the API:PVP 1:1 dispersion product disintegrate over the course of the dissolution run.
Besides the two dispersion products mentioned above, all the tablets disintegrate within five minutes of the dissolution run.
All of the dispersions containing a 4:1 API:polymer content show higher dissolution than their respective 1:1 API:polymer dispersions.

Based on the above, it could be expected that the API alone would display the highest overall dissolution; however, because this was not observed, the presence of polymers in the formulation appears to be helpful in promoting dissolution of the API in 0.1N HCl.

Based on the dissolution results discussed above, it can be concluded that the 4:1 API:polymer dispersions show higher overall dissolution than their respective 1:1 API:polymer dispersions. Despite the 4:1 API:HPMC and API:PVP dispersions showing higher dissolution than the 4:1 API:HPMCAS dispersion, the 4:1 API:HPMCAS dispersion was taken forward for additional formulation development. HPMCAS has already been dosed to dogs and has shown adequate absorption as a 1:1 API:HPMCAS dispersion. The 4:1 API:HPMC and API:PVP dispersions will be evaluated once formulation options are narrowed down using the 4:1 API:HPMCAS dispersion.

### Example 3

### Drug Product Preparations

An additional spray dried dispersion is prepared at 4:1 API:HPMCAS using the same parameters as described above.

Formulation Blend and Tablet Preparations

The following four surfactants are screened at 5% and 10% of the total formulation blend:

### 1) Vitamin E TPGS

### 2) Gelucire 44/14

### 3) Poloxamer 188 (Lutrol F68)

### 4) Sodium Lauryl Sulfate (SLS)

Because the Vitamin E TPGS and Gelucire 44/14 are semi-solids at room temperature and not provided in loose powder form, melt granulations are prepared at a 20 g scale for each surfactant containing 80% MCC (Avicel PH101) and 20% surfactant (the API dispersion is added at a later blending step). For each melt granulation, the granules are prepared by first melting the surfactant alone. Once the surfactant is in a molten state, it is added dropwise to the MCC powder bed while mixing at impeller and chopper speeds of 1000 RPM and 2500 RPM, respectively. The resulting granules are allowed to dry at room temperature. Following drying, the granules are sieved through a 250 μm sieve screen and stored in glass vials to be used for formulation blend preparations. For the poloxamer and SLS surfactants, no granulations are prepared since these two surfactants can be added directly to the formulation blend in loose powder form.

A 1 g formulation blend is prepared for each surfactant type/concentration with a target tablet weight of 250 mg. Blends are prepared in glass vials and hand mixed for 1 minute prior to tableting. Tables I through VIII list the formulation components and composition for each blend.

TABLE I
Formulation Blend with 10% Vitamin E TPGS

| Component | % of Blend | Amt/Capsule, mg |
|---|---|---|
| API:HPMCAS 4:1 SDD | 25.77% | 64.4 |
| Croscarmellose Sodium | 5.00% | 12.5 |
| Vit E TPGS/Avicel | 50.00% | 125.0 |
| Granulation | | |
| MCC | 19.23% | 48.1 |
| Total | 100.00% | 250.0 |

TABLE II
Formulation Blend with 5% Vitamin E TPGS

| Component | % of Blend | Amt/Capsule, mg |
|---|---|---|
| API:HPMCAS 4:1 SDD | 25.77% | 64.4 |
| Croscarmellose Sodium | 5.00% | 12.5 |
| Vit E TPGS/Avicel | 25.00% | 62.5 |
| Granulation | | |
| MCC | 44.23% | 110.6 |
| Total | 100.00% | 250.0 |

TABLE III
Formulation Blend with 10% Gelucire 44/14

| Component | % of Blend | Amt/Capsule, mg |
|---|---|---|
| API:HPMCAS 4:1 SDD | 25.77% | 64.4 |
| Croscarmellose Sodium | 5.00% | 12.5 |
| Gelucire/Avicel Granulation | 50.00% | 125.0 |
| MCC | 19.23% | 48.1 |
| Total | 100.00% | 250.0 |

TABLE IV
Formulation Blend with 5% Gelucire 44/14

| Component | % of Blend | Amt/Capsule, mg |
|---|---|---|
| API:HPMCAS 4:1 SDD | 25.77% | 64.4 |
| Croscarmellose Sodium | 5.00% | 12.5 |
| Gelucire/Avicel Granulation | 25.00% | 62.5 |
| MCC | 44.23% | 110.6 |
| Total | 100.00% | 250.0 |

TABLE V
Formulation Blend with 10% Poloxamer 188

| Component | % of Blend | Amt/Capsule, mg |
|---|---|---|
| API:HPMCAS 4:1 SDD | 25.77% | 64.4 |
| Croscarmellose Sodium | 5.00% | 12.5 |
| Poloxamer 188 | 10.00% | 25.0 |
| MCC | 59.23% | 148.1 |
| Total | 100.00% | 250.0 |

TABLE VI
Formulation Blend with 5% Poloxamer 188

| Component | % of Blend | Amt/Capsule, mg |
|---|---|---|
| API:HPMCAS 4:1 SDD | 25.77% | 64.4 |
| Croscarmellose Sodium | 5.00% | 12.5 |
| Poloxamer 188 | 5.00% | 12.5 |
| MCC | 64.23% | 160.6 |
| Total | 100.00% | 250.0 |

TABLE VII
Formulation Blend with 10% SLS

| Component | % of Blend | Amt/Capsule, mg |
|---|---|---|
| API:HPMCAS 4:1 SDD | 25.77% | 64.4 |
| Croscarmellose Sodium | 5.00% | 12.5 |
| SLS | 10.00% | 25.0 |
| MCC | 59.23% | 148.1 |
| Total | 100.00% | 250.0 |

TABLE VIII
Formulation Blend with 5% SLS

| Component | % of Blend | Amt/Capsule, mg |
|---|---|---|
| API:HPMCAS 4:1 SDD | 25.77% | 64.4 |
| Croscarmellose Sodium | 5.00% | 12.5 |
| SLS | 5.00% | 12.5 |
| MCC | 64.23% | 160.6 |
| Total | 100.00% | 250.0 |

For each formulation blend, 2 tablets are pressed at ˜250 mg fill weight for a target dosage strength of 50 mg (uncorrected for HCl salt content, ROI, and purity). A formulation blend aliquot is weighed and added to a single station tablet press fit with 5/16″ tooling. Each blend aliquot is pressed to ˜800 psi and the resulting tablets are stored in glass vials to be used for dissolution evaluations.

### Example 4

### Dissolution Evaluation

For each formulation, the 2 tablets prepared above are tested by dissolution according to the following dissolution parameters: 100 RPM paddle speed and 37.0° C. using USP apparatus 2 (Paddles) in 900 mL of 0.1N HCl. A 3 mL aliquot is removed at 15, 30, 45, 60, and 75 minutes from each vessel and filtered through a 0.45 μm nylon filter into an HPLC vial, discarding the first 2 mL of filtrate. After 60 minutes, the paddle speed is increased to 200 RPM. Samples are quantitated based on a standard prepared at ˜50 μg/mL. The average dissolution is calculated for the duplicate samples of each formulation. The dissolution profile for each formulation is shown in FIG. 2.

In conclusion, it has been observed that:
The tablets containing SLS show significant decrease in dissolution relative to the tablets containing no surfactant, despite full disintegration observed within 1 minute for all tablets.
No significant difference in dissolution is observed between the tablets containing variable amounts of Vitamin E TPGS and the tablets containing no surfactant.
The tablets containing Gelucire and poloxamer both show increases in dissolution, relative to the tablets containing no surfactant, with the tablets containing 10% Gelucire showing the highest overall dissolution and dissolution rate.

In the initial surfactant screening, Gelucire 44/14 shows the highest dissolution, so additional formulations are prepared with increased Gelucire concentration relative to the API within the formulation blend. The 5% and 10% Gelucire formulations correspond to an API:Gelucire concentration of 1:4 and 1:2, respectively.

### Example 5

### Drug Product Preparations

### Part 2

The same components described above are used to prepare 1 g formulation blends of the following:

1) Formulation Blend with No Surfactant
2) Formulation Blend with 1:1.5 API:Gelucire (14% Gelucire and 69% granulation)
3) Formulation Blend with 1:1.5 API:Gelucire (12% Gelucire and 58% granulation)
4) Formulation Blend with 1:1 API:Gelucire (13% Gelucire and 63% granulation)

For formulations 3 and 4, the target fill weight is increased to add MCC not granulated with Gelucire. The MCC is added to aid in tableting because issues can arise during automated tableting (sticking or picking) if all the MCC is added to the tablet in granulated form with Gelucire. Tables IX through XII list the formulation components and composition for each blend.

TABLE IX
Formulation Blend with No Surfactant

| Component | % of Blend | Amt/Capsule, mg |
|---|---|---|
| API:HPMCAS 4:1 SDD | 25.77% | 64.4 |
| Croscarmellose Sodium | 5.00% | 12.5 |
| MCC | 69.23% | 173.1 |
| Total | 100.00% | 250.0 |

TABLE X
Formulation Blend with 14% Gelucire 44/14 and 69% Granulation
(1:1.5 API:Gelucire)

| Component | % of Blend | Amt/Capsule, mg |
|---|---|---|
| API:HPMCAS 4:1 SDD | 25.77% | 64.4 |
| Croscarmellose Sodium | 5.00% | 12.5 |
| Gelucire/Avicel Granulation | 69.23% | 173.1 |
| Total | 100.00% | 250.0 |

TABLE XI
Formulation Blend with 12% Gelucire 44/14 and 58% Granulation
(1:1.5 API:Gelucire)

| Component | % of Blend | Amt/Capsule, mg |
|---|---|---|
| API:HPMCAS 4:1 SDD | 21.64% | 64.4 |
| Croscarmellose Sodium | 5.00% | 14.9 |
| Gelucire/Avicel Granulation | 58.13% | 173.1 |
| MCC | 15.22% | 45.3 |
| Total | 100.00% | 297.7 |

TABLE XII
Formulation Blend with 13% Gelucire 44/14 and 63% Granulation
(1:1 API:Gelucire)

| Component | % of Blend | Amt/Capsule, mg |
|---|---|---|
| API:HPMCAS 4:1 SDD | 16.11% | 64.4 |
| Croscarmellose Sodium | 5.00% | 20.0 |
| Gelucire/Avicel Granulation | 62.50% | 250.0 |
| MCC | 16.39% | 65.6 |
| Total | 100.00% | 400.0 |

For each formulation blend, 2 tablets are pressed at their respective fill weight for a target dosage strength of 50 mg (uncorrected for HCl salt content, ROI, and purity). A formulation blend aliquot is weighed and pressed using a single station tablet press. For the formulations pressed at 250 mg and 297.7 mg fill weights, 5/16″ (0.3135″) tooling is used. For the formulation pressed at 400 mg fill weight, 0.3437″ tooling is used. Each blend aliquot is pressed to ˜800 psi and the resulting tablets are stored in glass vials to be used for dissolution evaluations as described above.

### Example 6

### Dissolution Evaluation

### Part 2

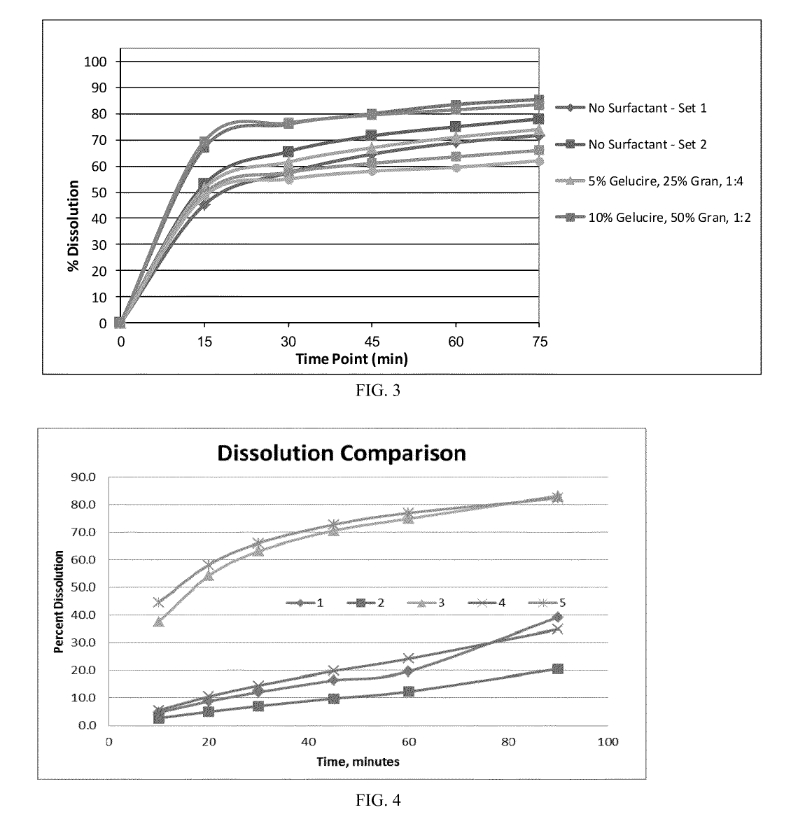

The dissolution profile for the two formulations containing no surfactant and all formulations containing Gelucire are shown in FIG. 3.

In conclusion, it has been observed that:
When comparing the two formulations containing no surfactant, the formulation containing the initial 4:1 API:HPMCAS dispersion prepared shows ˜8% less dissolution at each time point.
The 10% Gelucire (1:2 Gelucire:API) and 13% Gelucire (1:1 Gelucire:API) show the highest overall dissolution. Since the 10% Gelucire contains less granulation in the overall formulation, 10% Gelucire would be used for future formulation development.

Since tablets containing poloxamer 188 show increased dissolution relative to tablets containing no surfactant (see FIG. 2), additional formulations are prepared at 2% and 15% poloxamer for testing by dissolution. When comparing the results in FIG. 2, the 10% Gelucire and 5% poloxamer 188 formulations show the highest overall dissolution, so a formulation is prepared containing both surfactants at their respective concentrations and is tested by dissolution.

Because the API:PEG dispersion shows one of the highest overall dissolutions, an additional formulation is prepared using PEG1450 in a loose powder form at 10% of the formulation blend and is tested by dissolution.

The following formulations are identified for assessment in an animal study:

1) Current GLP formulation—Capsule Drug Product
To serve as the control
   2) Current GLP formulation—Tablet Drug Product
To compare absorption of NGP555 in capsule vs. tablet form
   3) Formulation with 4:1 API:HPMCAS
To compare absorption of 1:1 NPG555:HPMCAS vs. 4:1 NGP555:HPMCAS
   4) Wild Card—Formulation may contain the following:
1:1 API:HPMCAS with surfactant
4:1 API:HPMCAS with surfactant
4:1 API:polymer
4:1 API:polymer with surfactant

In certain embodiments, the current GLP formulation will contain two additional excipients to aid in automated manufacturing for human clinical trials. These two additional excipients, a flow aid (e.g., silicon dioxide) and a lubricant (e.g., magnesium stearate), can be considered inert and should not affect the absorption of NGP555.

To further assist in formulation development for NGP555, the amorphous solid dispersion formulation containing NGP555 spray dried with HPMC-AS and mixed with microcrystalline cellulose and croscarmellose sodium is processed into 5 different final dosage forms. All five are evaluated for in-vitro performance using a discriminating dissolution test. Three are then selected for in-vivo evaluation in dogs based on in-vitro performance.

Starting Material

The GLP formulation lot 1212-5-75-1 (composition summarized in the following table) is used as the starting material for each dosage form.

TABLE
Lot 1212-5-75-1 Composition

| Component | Percent (wt/wt) |
|---|---|
| NGP555 | 20% |
| HPMC-AS | 20% |
| Avicel PH101 (MCC) | 55% |
| Croscarmellose Sodium | 5% |

### Example 7

### Drug Product Presentation 1

Formulation 1212-5-75-1 is manually filled into size 0 white gelatin capsules using a profile apparatus. The target fill weight is 250 mg for an active dose of 50 mg. The manual filling process results in uniform fill weights with a weight variation of 1.5%. This presentation is the simplest of the five.

### Drug Product Presentation 2

Formulation 1212-5-75-1 is directly compressed into tablets using a single station hydraulic press. Standard round concave tooling is used, with a compression force of 1000 psi. Tablet properties are listed in the following Table.

TABLE
Tablet Properties

| | |
|---|---|
| Parameter | Comment |

| | | |
|---|---|---|
| Gross Weight | 250 | mg |
| Active Strength | 50 | mg |
| Tooling | 5/16 | in. Round Concave |
| Compression Force | 1000 | psi |
| Height | 5.1 | mm |
| Breaking Force | 14.0 | kp |

### Roller Compaction

For the production of the additional 3 dosage forms, roller compacted material is used. Approximately 500 grams of the bulk formulation 1212-5-75-1 is roller compacted and milled according to the parameters set forth in the following Table. This single batch is used for the preparation of the final three dosage forms, and is referred to herein as the “roller-compacted formulation”.

TABLE
Roller Compaction and Milling

| Parameter | Comment |
|---|---|
| Roller Compactor | Vector TFC Micro Roller |
| Roller Speed | 2.010 RPM |
| Screw Speed | 33.00 RPM |
| Roll Current | 0.4 V |
| Screw Current | 0.65 V |
| Co-Mill | Quadro 197S |
| Screen | Round Flat 1143 μm |
| Speed | 2500 RPM |
| Approximate Process Yield | 80% |

### Example 8

### Drug Product Presentation 3

The roller-compacted formulation is filled into size 1 white gelatin capsules using a profill apparatus. The target fill weight is 250 mg for an active strength of 50 mg. The filling process results in a uniform fill with a weight variation of 1.5%. This presentation is the simplest of the three roller compacted formulations. This fill is achieved with a single flood fill without compression. Size 2 may be more appropriate for GMP manufacturing by this method.

### Drug Product Presentation 4

Roller compacted formulation is directly compressed into tablets using a single station hydraulic press. Standard round concave tooling is used, with a compression force of 1500 psi. Tablet properties are summarized in the following Table.

Note that despite the increase in compression force, the tablets have a lower hardness/breaking force. In order to understand the effect of double compression (ribbon formation plus tablet formation), a tablet is compressed at 1000 psi to mimic Drug Product Presentation 2. This tablet has a breaking force of 5.2 kp. This indicates a significant loss in compressibility due to the roller compaction process, which tends to decrease disintegration and dissolution performance.

TABLE
Tablet Properties

| Parameter | Comment | |
|---|---|---|
| Gross Weight | 250 | mg |
| Active Strength | 50 | mg |
| Tooling | 5/16 | in. Round Concave |
| Compression Force | 1500 | psi |
| Height | 4.9 | mm |
| Breaking Force | 8.9 | kp |

### Example 9

### Drug Product Presentation 5

This formulation presentation is made to address the observations referred to above for Presentation 4, regarding the loss of formulation compressibility and its impact on performance. The roller compacted formulation is mixed with additional extra-granular excipients equivalent to 20% of the final drug product weight. The final composition of the formulation is detailed in the following Table.

TABLE
Presentation 5 Composition

| Component | Percent (wt/wt) | Process Step |
|---|---|---|
| NGP555 | 16% | Roller |
| HPMC-AS | 16% | Compacted |
| Avicel PH101 (MCC) | 44% | |
| Croscarmellose Sodium | 4% | |
| Avicel PH102 (MCC) | 15% | Extra Granular |
| Croscarmellose Sodium | 5% | |

This formulation is then compressed into tablets using a single station hydraulic press. Standard round concave tooling is used with a compression force of 1600 psi. When compressed into the 5/16 inch tooling with 1000 psi, the corresponding tablet breaking force is 9.3 kp. This indicates that the additional extra-granular MCC improves compressibility. However, these tablets have an awkward aspect ratio due to the increased unit weight. ⅜ inch tooling is used for the final compressed dosage form. The tablet properties are detailed in the following Table.

TABLE
Tablet Properties

| Parameter | Comment | |
|---|---|---|
| Gross Weight | 312.5 | mg |
| Active Strength | 50 | mg |
| Tooling | ⅜ | in. Round Concave |
| Compression Force | 1600 | psi |
| Height | 4.5 | mm |
| Breaking Force | 9.9 | kp |

### Example 10

### Dissolution Testing

All five of the above-described formulations are tested for dissolution by the parameters detailed in the following Table.

TABLE
Dissolution Parameters

| Parameter | Comment |
|---|---|
| Apparatus | Paddles, USP No. 2 w/ Sinkers for Capsules |
| Media | 0.1N HCl |
| Volume | 900 mL |
| Temperature | 37.0° C. |
| Paddle Speed | 75 RPM from 0-60 Minutes |
| | 200 RPM from 60-90 Minutes |
| Pull Times | 10, 20, 30, 45, 60, & 90 (∞) Minutes |
| Units | n = 6 |
| Sample Procedure | VK8000 Auto-Sampler Directly into HPLC Vials |
| Filters | QLA Porous Micron Filters, 1 Micron Pore Size |

The dissolution results are provided in the following Table and FIG. 4.

TABLE
Dissolution Results

| | Formulation Presentation/ | | | | |
|---|---|---|---|---|---|
| Time, | Percent Dissolution | | | | |
| minutes | 1 | 2 | 3 | 4 | 5 |
| 10 | 4.6 | 2.5 | 37.6 | 5.4 | 44.6 |
| 20 | 8.7 | 4.9 | 54.1 | 10.4 | 58.2 |
| 30 | 12.0 | 6.9 | 63.1 | 14.5 | 66.0 |
| 45 | 16.3 | 9.7 | 70.5 | 19.7 | 72.7 |
| 60 | 19.5 | 12.3 | 74.9 | 24.2 | 77.0 |
| 90 | 39.2 | 20.5 | 83.1 | 34.8 | 82.4 |

Presentations 1, 2, and 4 are observed to have very poor disintegration. The release occurred primarily by slow erosion of the dosage unit. In each case, a plug of powder was present after the 60 minute time point.

In contrast, presentation 3 was observed to disintegrate very quickly, with complete disintegration occurring for all units between 2 and 6 minutes. Even more dramatically, the disintegration of Presentation 5 was instantaneous. For all vessels, the entire tablet was disintegrated with the undissolved powder evenly suspended in the dissolution bath in less than 60 seconds.

In summary it can be concluded that:
1—The current NGP555 formulation has very poor disintegration and dissolution properties that require secondary processing.
2—Roller compaction greatly improves the performance when filled directly into capsules.
3—Roller compaction significantly reduces the compressibility of the formulation, and results in tablets with poor disintegration.
4—The addition of 20% extra-granular excipients improves the compressibility of the roller compacted formulation and results in tablets with extremely rapid disintegration.

### Example 11

### Exemplary Manufacturing Protocol

### Solution Preparation

The procedure employed for solution preparation was as follows:
1. Weight 8095 grams of methanol into the 15-L stainless steel stock pot.
2. Begin mixing at a speed sufficient to produce a medium vortex.
3. Sieve the API through a 1000 μm screen, and add 472.34 grams of sieved API to the methanol while mixing.
4. Sieve the HPMCAS through a 1000 μm screen, and add 422.42 grams of sieved HPMCAS to the methanol while mixing.
5. Cover the pot with multiple layers of foil and allow mixing overnight.

### Spray Drying

Spray drying was carried out according to the parameters set forth in the following table:

TABLE
Spray Parameters

| Parameter | Set Point/Range |
|---|---|
| Aspirator (CCDA) | 100% (11-13.5 SCFM) |
| Atomizing Air (Nitrogen) | 45 mm (~550 L/hr) |
| Inlet Temperature | 155° C.-170° C. |
| Outlet Temperature | 60° C.-70° C. |
| Chiller Temp | −20° C. |
| Filter Pulse | 2 |

The spraying procedure employed is as follows:
1—Engage the Aspirator, the atomizing air, and the heater.
2—Calibrate the external pump to 25 g/minute.
   a. Note the L16 tubing runs through the pump and the smaller tubing is secured to the spray nozzle.
3—Once the outlet temperature is >85° C., begin spraying pure methanol for ˜1 minute.
4—Switch to the dispersion after priming the nozzle with methanol.
   a. Failure to prime with methanol before starting and stopping will likely result in a clogged nozzle.
5—Adjust the inlet temperature as needed to maintain the target outlet temperature.
6—Spray until one of the following occurs:
   a. 1.5 kg of solution has been sprayed (or 1 hour of spray time has elapsed).
   b. An inlet temperature of >170° C. is required to maintain an outlet temperature of ≧60° C.
   c. The solvent trap is 95% full.
7—Switch back to pure methanol for ˜1 minute to clear the tubing lines and gun.
8—Exchange the filter sock, empty the product collection flasks, and reset the inlet temperature to 155° C.
9—Repeat 3-8 until all the solution has been sprayed.

### Drying:

Spread the dispersion evenly over 4 trays in the vacuum oven. Set the oven to 40° C. and pull full vacuum. Leave overnight. Pull a 1-2 gram sample from a random spot on Trays 1 and 3. Run LOD on each sample. Average the results and determine theoretical drug load using Equation 1.

### Mixing

The procedure employed for mixing was as follows:
1—Tare the Blending Vessel, and transfer all the dispersion into the vessel. Determine the weight of dispersion available for mixing.
2—Determine the total batch size by Equation 2.

Total Batch Size=Weight of Dispersion*Drug Load(Eq.1)*460 mg/100 mg (Eq. 2)
3—Determine the target amount of Ac-di-sol by Equation 3.

Ac-di-sol Wt=Total Batch Size*0.05 (Eq. 3)
4—Determine the target amount of Mg Stearate by Equation 4.

Mg Stearate Wt=Total Batch Size*0.01 (Eq. 4)
5—Determine the target amount of Avicel PH101 by Equation 5.

Avicel Wt=Total Batch Size−Summed Wt of Dispersion,Ac-di-sol,&Mg Stearate (Eq. 5)
6—Add the target amounts of Avicel and Ac-di-sol to the blend vessel.
7—Engage the mixer for 10 minutes on the second set of pulleys.
8—Sieve all material through a 1000 μm screen, return to the vessel, and mix for an additional 10 minutes.
9—Pull an IPT sample from the top, middle, and bottom of the vessel using a sterile plastic sample thief.
10—Confirm IPT Results (Mean 95-105%, Individual 90-110%)
11—Sieve the Mg Stearate through a 1000 μm screen.
12—Weigh the target amount into the blending vessel and mix for an additional 2 minutes.

### Roller Compaction

The parameters employed for roller compaction are summarized in the following table:

TABLE
Roller Compaction Parameters

| Parameter | Set Point | |
|---|---|---|
| Pressure | 6000 | psi |
| Roll Speed | 5 | RPM |
| Screw Speed | 30 | RPM |
| Expected Feed Rate | 7.5-8.0 | Kg/hr |
| Expected Roll Time | <1 | hour |

Throughout the process, the ribbon and any leaked powder is transferred to a 1000 μm sieve and pan. The ribbons are shaken to knock loose any uncompressed powders. This powder is then fed back into the hopper to be compressed into a ribbon.

### Density Measurement:

Three sections of ribbon are randomly selected, and broken down to an approximate length of 6-10 cm. The length, depth, thickness, and weight are measured. The envelop density is then determined by Equation (1). This result is FIO.

Envelop Density, g/cc=wt, g/(length, cm*depth, cm*thickness, cm) Equation (1)

### Milling

Milling is carried out according to the following parameters:

Speed Set Point: 50%, 2650 RPM

Spacer: 125/1000 inch

Screen: 1016 grated

Blade: Square

### Mixing/IPT:

The milled material is returned to the original blending vessel, the weight of milled material determined, and then mixed for 5 minutes. After 5 minutes, IPT samples are pulled from the top, middle, and bottom using a fresh sample thief. Hold for IPT results. If specifications are met (Mean 95-105%, Individual 90-110%), proceed with splitting the batch and filling capsules.

### Batch Segregation and Capsule Filling, 100 Mg Capsules

The weight of material to charge to the 25 mg production is determined by Equation (2):

Charge Weight,25 mg Batch=Total Weight of Final Blend*7/41 Equation (2)

All remaining material is filled to generate 100 mg capsules employing the parameters set forth in the following table.

TABLE
Filling Parameters

| Parameter | Set Point |
|---|---|
| Operation | Profill (n = 100) |
| Size | 0 |
| Capsules | White Gelatin (Coni-Snaps) |
| Fill Weight | 460 mg/Unit |
| Tray Weight | 46.46 grams/Tray |
| Weight Check | AQL (n = 13) |
| Upper Weight Limit | 494.5 + Shell Weight, mg |
| Lower Weight Limit | 425.5 + Shell Weight, mg |

### Blending, 25 mg Capsules

The procedure: employed for blending is as follows:
1—Batch size is determined by Equation (3).

Batch Weight, g=Weight of Roller Compacted Powder*340/115 Equation (3)
where:
   340=the 25 mg fill weight, and
   115=RC Formulation weight/capsule
2—Determine the required amount of Ac-Di-Sol by Equation (4).

Wt of Ac-di-sol=Batch Weight*11.25/340 Equation (4)
3—Determine the required amount of Mg Stearate by Equation (5).

Wt of Mg Stearate=Batch Weight*2.25/340 Equation (5)
4—Determine the required amount of Avicel by Equation (6).

Wt of Avicel=Batch Weight*211.5/340 Equation (6)
5—Sieve the Avicel, and Ac-di-sol through a 1000 μm sieve.
6—Weigh the Roller Compacted formulation, Avicel, and Ac-di-sol into the 4-L vessel.
7—Mix for 10 minutes on the turbula mixer engaged using the second set of pulleys.
8—Pull IPT sample samples from the top, middle, and bottom using the sterile plastic sample thief.
9—Confirm IPT Results (Mean 95-105%, Individual 90-110%)
10—Sieve the Mg Stearate through a 1000 μm screen.
11—Weigh the target amount into the blending vessel and mix for an additional 2 minutes.

### Capsule Filling, 25 Mg Capsules

Fill all remaining material to generate 25 mg capsules employing the following filling parameters:

TABLE
Filling Parameters

| Parameter | Set Point |
|---|---|
| Operation | Profill (n = 100) |
| Size | 0 |
| Capsules | White Gelatin (Coni-Snaps) |
| Fill Weight | 340 mg/Unit |
| Tray Weight | 34.34 grams/Tray |
| Weight Check | AQL (n = 13) |
| Upper Weight Limit | 365.5 + Shell Weight, mg |
| Lower Weight Limit | 314.5 + Shell Weight, mg |

### Bottle Filling

The bottle filling requirements are the same for both lots.

Count: 15 units

Bottle: 60 cc HDPE

Desiccant: 1 gram Sorbit Packet

Closure: CRC with Induction Seal

Label: Standard Ptek Format unless otherwise specified by Client.

Any capsules remaining that are not sufficient to make a 30 count bottle, may be double bagged with desiccant and transferred to development.

Although the invention is illustrated and described herein with reference to specific embodiments, the invention is not intended to be limited to the details shown. Rather, various modifications may be made in the details within the scope and range of equivalents of the claims without departing from the invention.

That which is claimed is:

1. A formulation suitable for delivery of gamma secretase modulators (GSMs) to a subject in need thereof, said formulation comprising one or more of (a), (b) and/or (c) as follows:
(a) a finely divided form of said GSM; and/or
(b) a substantially amorphous form of said GSM; and/or
(c) one or more GSM(s) in the further presence of one or more excipients therefor.

2. A formulation according to claim 1 wherein said gamma secretase modulator is a compound of Formula (I) having the structure:
(A)-L_{A}-(B)-L_{B}-(C)-L_{C}-(D) (I)
as well as analogs, homologs, prodrugs, derivatives, and pharmaceutically acceptable salts thereof,
wherein:
A is an optionally substituted 1,3-imidazole or an optionally substituted 1,2,3-triazole having the structure: 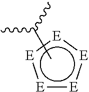
wherein E at positions 1 and 3 are N, E at position 2 is N or CH, E at position 4 is CR¹, and E at position 5 is CH;
each R¹ is hydrogen, halogen, substituted or unsubstituted alkyl, substituted or unsubstituted alkenyl, substituted or unsubstituted alkynyl, substituted or unsubstituted alkoxy, substituted or unsubstituted alkylamido, substituted or unsubstituted alkylamino, substituted or unsubstituted amino, substituted or unsubstituted cycloalkyl, or substituted or unsubstituted aryl;
B is an optionally substituted phenyl, an optionally substituted pyridyl or an optionally substituted pyrimidinyl having the structure: 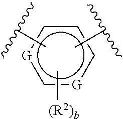
wherein each G is independently CR² or N;
each R² is independently selected from hydrogen, halogen, substituted or unsubstituted alkyl, substituted or unsubstituted alkenyl, substituted or unsubstituted alkynyl, substituted or unsubstituted alkoxy, substituted or unsubstituted alkylamido, substituted or unsubstituted alkylamino, or substituted or unsubstituted amino; and
b is 0-2;
C is an optionally substituted thiazole having the structure: 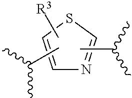
wherein R³ is hydrogen, halogen, substituted or unsubstituted alkyl, substituted or unsubstituted cycloalkyl, substituted or unsubstituted aryl, substituted or unsubstituted heteroaryl, or substituted or unsubstituted alkoxy;
D is an optionally substituted aryl having the structure:
wherein each R⁵ is independently selected from hydrogen, halogen, substituted or unsubstituted alkyl, substituted or unsubstituted alkenyl, substituted or unsubstituted alkynyl, substituted or unsubstituted alkoxy, substituted or unsubstituted cycloalkyl, substituted or unsubstituted heterocycle, substituted or unsubstituted aryl, substituted or unsubstituted heteroaryl, or substituted or unsubstituted amino, or substituted or unsubstituted alkylamino; and
d is 0-5;
L_{A} is a covalent bond;
L_{B} is a covalent bond; and
L_{C} is —NR—.

3. The formulation of claim 1 wherein said formulation provides enhanced GSM transport across the blood-brain barrier.

4. The formulation of claim 1 wherein said formulation provides enhanced GSM transport from the gut to the bloodstream.

5. The formulation of claim 1 wherein said formulation achieves improved compound stability.

6. The formulation of claim 2 wherein said GSM has a structure corresponding to Formula (II):

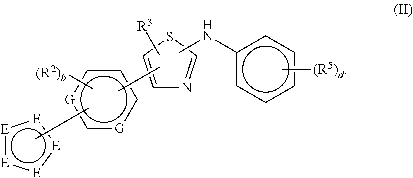

7. The formulation of claim 2 wherein said GSM has a structure corresponding to Formula (III):

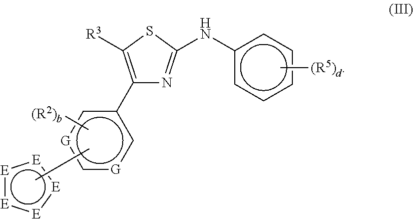

8. The formulation of claim 2 wherein said GSM has a structure corresponding to Formula (IV):

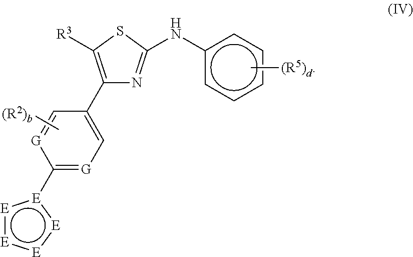

9. The formulation of claim 2 wherein said GSM has a structure corresponding to Formula (V):

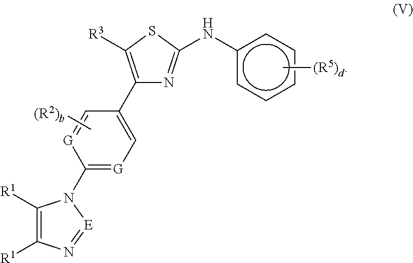

10. The formulation of claim 2 wherein said gamma secretase modulator is selected from the group consisting of those compounds set forth in paragraph [0055] hereof.

11. A method of making a gamma secretase modulator (GSM) suitable for delivery to a subject in need thereof, said method comprising subjecting said GSM to one or more of the following:
(a) finely dividing said GSM to a minimum/maximum particle size(s) in the range of 25-106 μM, with a particle size distribution in the range of 5 μM-109 μM; and/or
(b) converting said GSM to substantially amorphous form; and/or
(c) combining said GSM with one or more excipients therefor:

12. The method of claim 11 wherein said GSM is a compound of Formula (I) having the structure:
(A)-L_{A}-(B)-L_{B}-(C)-L_{C}-(D) (I)
as well as analogs, homologs, prodrugs, derivatives, and pharmaceutically acceptable salts thereof,
wherein:
A is an optionally substituted 1,3-imidazole or an optionally substituted 1,2,3-triazole having the structure: 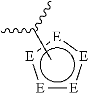
wherein E at positions 1 and 3 are N, E at position 2 is N or CH, E at position 4 is CR¹, and E at position 5 is CH;
each R¹ is hydrogen, halogen, substituted or unsubstituted alkyl, substituted or unsubstituted alkenyl, substituted or unsubstituted alkynyl, substituted or unsubstituted alkoxy, substituted or unsubstituted alkylamido, substituted or unsubstituted alkylamino, substituted or unsubstituted amino, substituted or unsubstituted cycloalkyl, or substituted or unsubstituted aryl;
B is an optionally substituted phenyl, an optionally substituted pyridyl or an optionally substituted pyrimidinyl having the structure: 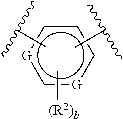
wherein each G is independently CR² or N;
each R² is independently selected from hydrogen, halogen, substituted or unsubstituted alkyl, substituted or unsubstituted alkenyl, substituted or unsubstituted alkynyl, substituted or unsubstituted alkoxy, substituted or unsubstituted alkylamido, substituted or unsubstituted alkylamino, or substituted or unsubstituted amino; and
b is 0-2;
C is an optionally substituted thiazole having the structure: 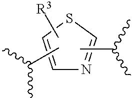
wherein R³ is hydrogen, halogen, substituted or unsubstituted alkyl, substituted or unsubstituted cycloalkyl, substituted or unsubstituted aryl, substituted or unsubstituted heteroaryl, or substituted or unsubstituted alkoxy;
D is an optionally substituted aryl having the structure: 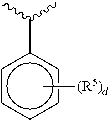
wherein each R⁵ is independently selected from hydrogen, halogen, substituted or unsubstituted alkyl, substituted or unsubstituted alkenyl, substituted or unsubstituted alkynyl, substituted or unsubstituted alkoxy, substituted or unsubstituted cycloalkyl, substituted or unsubstituted heterocycle, substituted or unsubstituted aryl, substituted or unsubstituted heteroaryl, or substituted or unsubstituted amino, or substituted or unsubstituted alkylamino; and
d is 0-5;
L_{A} is a covalent bond;
L_{B} is a covalent bond; and
L_{C} is —NR—.

13. A formulation prepared by the method of claim 11.

14. A formulation according to claim 13 which is prepared by hot melt extrusion.

15. A formulation according to claim 13 which is prepared by co-precipitation.

16. A formulation according to claim 13 which is prepared by spray drying.

17. A method for delivering a gamma secretase modulators (GSM) to a subject in need thereof, said method comprising administering an effective amount of a formulation according to claim 13 to said subject.

18. A method for delivering a gamma secretase modulators (GSM) to a subject in need thereof, said method comprising:
subjecting said GSM to one or more of the following:
(a) finely dividing said compound to a minimum/maximum particle size(s) in the range of 25 μM-106 μM, with a particle size distribution in the range of 5 μM-109 μM; and/or
(b) converting said compound to substantially amorphous form; and/or
(c) combining said compound with one or more excipients therefor: and thereafter
administering an effective amount thereof to said subject.
